(19) United States
(12) Patent Application Publication
(10) Pub. No.: US 2020/0333324 A1
(43) Pub. Date: Oct. 22, 2020

(54) METHOD OF PREDICTING CLINICAL OUTCOME OF ANTICANCER AGENTS

(71) Applicant: MITRA RXDX, INC., Woburn, MA (US)

(72) Inventors: James P. CASSIDY, Woburn, MA (US); Aaron GOLDMAN, Woburn, MA (US); Pradip K. MAJUMDER, Woburn, MA (US); Padhma D. RADHAKRISHNAN, Woburn, MA (US)

(21) Appl. No.: 16/484,385

(22) PCT Filed: Feb. 7, 2018
(86) PCT No.: PCT/US2018/017299

§ 371 Date: Aug. 7, 2019

Related U.S. Application Data
(60) Provisional application No. 62/456,550, filed on Feb. 8, 2017.
(60) Provisional application No. 62/464,993, filed on Feb. 28, 2017.
(60) Provisional application No. 62/596,060, filed on Dec. 7, 2017.

(51) Int. Cl. G01N 33/50; C12N 5/09; G01N 33/574; G16B 20/00; G16B 40/00; G16C 20/30

(52) U.S. Cl. CPC G01N 33/5011; C12N 5/0694; G16C 20/30; G16B 20/00; G16B 40/00; G01N 33/574

(57) ABSTRACT

The invention provides methods of predicting responsiveness to a therapeutic agent for treating cancer in an individual using a tumor tissue culture capable of mimicking physiologically relevant signaling. In some embodiments, the therapeutic agent is an immunotherapeutic agent. In some embodiments, the methods are capable of distinguishing differential responsiveness to multiple therapeutics agents against the same target.

### CROSS-REFERENCE TO RELATED APPLICATIONS

This application claims priority from U.S. Provisional Patent Application No. 62/456,550, filed Feb. 8, 2017; U.S. Provisional Patent Application No. 62/464,993, filed Feb. 28, 2017; and U.S. Provisional Patent Application No. 62/596,060, filed Dec. 7, 2017, the contents of each of which are incorporated herein by reference in its entirety.

### TECHNICAL FIELD

This application pertains to prognostic and therapeutic methods involving determining the responsiveness of an individual having cancer to one or more therapeutic agents based on a clinical response predictor.

### BACKGROUND

Emerging clinical evidence using immunotherapy in recent years has demonstrated its power to suppress tumor growth by releasing the brakes on the immune system. For example, blockade of immune checkpoints, such as PD-1, has revolutionized treatment options for patients with aggressive cancers such as head and neck squamous cell carcinoma (HNSCC). However, clinical responses to PD-1 inhibition vary widely among patients. Additionally, multiple FDA-approved drugs against the same immune checkpoints have resulted in globally distinct outcomes in the clinic. There is a huge unmet need to understand these disparities at the individual patient level, and to maximize the clinical benefits of these agents.

### SUMMARY

In some embodiments, there is provided a method of predicting responsiveness to an immunotherapeutic agent for treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; and d) using the output to predict responsiveness of the individual to administration of the immunotherapeutic agent.

In some embodiments, there is provided a method of classifying likely responsiveness to an immunotherapeutic agent for treating cancer in an individual in need thereof, comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; and d) using the output to classify the likely responsiveness of the individual to administration of the immunotherapeutic agent.

In some embodiments, there is provided a computer-implemented method for predicting responsiveness to an immunotherapeutic agent for treating cancer in an individual in need thereof, the method comprising: a) accessing a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; and d) using the output to predict responsiveness of the individual to administration of the immunotherapeutic agent.

In some embodiments, according to any of the methods described above, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output.

In some embodiments, according to any of the methods described above, the output predicts complete clinical response, partial clinical response, or no clinical response of the individual to administration of the immunotherapeutic agent.

In some embodiments, according to any of the methods described above, the output predicts response or no response of the individual to administration of the immunotherapeutic agent.

In some embodiments, according to any of the methods described above, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

In some embodiments, according to any of the methods described above, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual.

In some embodiments, according to any of the methods described above, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform.

In some embodiments, according to any of the methods described above, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform.

In some embodiments, there is provided a method of selecting a preferred therapeutic agent for treating cancer in an individual in need thereof from among a plurality of therapeutic agents against the same target molecule, the method comprising: a) obtaining a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform; b) inputting the readout into a predictive model; c) using the predictive model to generate an output for each of the plurality of therapeutic agents; d) using the outputs to predict responsiveness of the individual to administration of each of the plurality of therapeutic agents, and e) selecting from among the plurality of therapeutic agents the therapeutic agent with the highest predicted responsiveness as the preferred therapeutic agent.

In some embodiments, according to any of the methods described above, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent.

In some embodiments, according to any of the methods described above, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

In some embodiments, according to any of the methods described above, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual.

In some embodiments, according to any of the methods described above, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform.

In some embodiments, according to any of the methods described above, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform.

In some embodiments, there is provided a method of treating cancer in an individual in need thereof, the method comprising administering to the individual an immunotherapeutic agent to which the individual is predicted to respond according to any of the methods described above. In some embodiments, the individual is predicted to have a complete clinical response or partial clinical response to administration of the immunotherapeutic agent.

In some embodiments, there is provided a method of treating cancer in an individual in need thereof, the method comprising administering to the individual a preferred therapeutic agent from among a plurality of therapeutic agents against the same target molecule, wherein the preferred therapeutic agent is selected according to any of the methods described above. In some embodiments, the individual is predicted to have a complete clinical response or partial clinical response to administration of the preferred therapeutic agent.

In some embodiments, according to any of the methods described above, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab.

In some embodiments, according to any of the methods described above, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab.

In some embodiments, there is provided a method of predicting responsiveness to an therapeutic agent for treating cancer in an individual in need thereof, the method comprising: a) obtaining a readout comprising assessment scores from a plurality of assays conducted on a tumor tissue culture, wherein the tumor tissue culture comprises i) a tumor microenvironment platform cultured with tumor tissue from the individual; and ii) the therapeutic agent; b) converting the readout into a sensitivity index; and c) using the sensitivity index to predict responsiveness to the therapeutic agent, wherein the therapeutic agent is an immunotherapeutic agent.

In some embodiments, there is provided a method of selecting a preferred therapeutic agent for treating cancer in an individual in need thereof from among a plurality of therapeutic agents against the same target molecule, the method comprising: a) obtaining a readout comprising assessment scores from a plurality of assays conducted on a tumor tissue culture, wherein the tumor tissue culture comprises i) a tumor microenvironment platform cultured with tumor tissue from the individual; and ii) one of the plurality of therapeutic agents; b) converting the readout of step a) into a sensitivity index; and c) using the sensitivity index of step b) to predict responsiveness to the therapeutic agent, wherein steps a), b) and c) are carried out sequentially for each of the plurality of therapeutic agents, and wherein the therapeutic agent with the highest sensitivity index that predicts responsiveness is selected as the preferred therapeutic agent.

In some embodiments, according to any of the methods described above, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

In some embodiments, according to any of the methods described above, the tumor microenvironment platform comprises an extracellular matrix composition comprising culture medium and one or more of collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, or autologous peripheral blood nuclear cells (PBNC).

In some embodiments, according to any of the methods described above, step a) further comprises culturing tumor tissue obtained from the individual with the tumor microenvironment platform and adding the therapeutic agent to the tumor microenvironment platform. In some embodiments, step a) further comprises conducting the plurality of assays on the tumor tissue culture to generate assessment scores, thereby producing the readout. In some embodiments, step b) further comprises multiplying the assessment score of each of the plurality of assays with a weightage score for the assay to obtain a weighted assay score for each of the plurality of assays; and combining the weighted assay scores for each of the plurality of assays to obtain the sensitivity index.

In some embodiments, according to any of the methods described above, the sensitivity index predicts complete clinical response, partial clinical response, or no clinical response to the therapeutic agent in the individual.

In some embodiments, there is provided a method of treating cancer in an individual in need thereof, the method comprising administering to the individual a therapeutic agent having a sensitivity index according to any of the methods described above that predicts responsiveness.

In some embodiments, there is provided a method of treating cancer in an individual in need thereof, the method comprising administering to the individual a preferred therapeutic agent from among a plurality of therapeutic agents against the same target molecule, wherein the preferred therapeutic agent is selected according to any of the methods described above.

In some embodiments, according to any of the method of treating cancer described above, the therapeutic agent has a sensitivity index that predicts complete clinical response or partial clinical response in the individual.

In some embodiments, according to any of the methods described above, the therapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab.

In some embodiments, according to any of the methods described above, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab.

In some embodiments, according to any of the methods described above, the individual is human.

### BRIEF DESCRIPTION OF THE DRAWINGS

FIG. 1 shows box plots for the results of analysis of baseline tumor tissue for percent of cells positive for Ki67, CD8, CD68, PD-1, PD-L1, ICOS, FOXP3, and pSTAT1 by IHC, and tumor content by H&E staining.

FIG. 2 shows IHC analysis for VEGFR, CD34, TGF-β, CD8, CD68, PDL1, FOXP3, IL6, IL8, CXCR4, and MMP-9 expression in tumor tissue cultured in the tumor microenvironment platform for 3 days (T3) compared to baseline tumor tissue at TO.

FIGS. 3A and 3B show results for H&E staining and IHC analysis for Ki67 and Caspase 3 expression in tumor tissue cultured in the tumor microenvironment platform treated with Pembrolizumab, Nivolumab, or IgG control for 3 days (T3) compared to baseline tumor tissue at TO. FIG. 3A shows results for tumor tissue derived from patient ID 2941 and FIG. 3B shows results for tumor tissue derived from patient ID 2942.

FIG. 3C shows quantification of the results from FIGS. 3A and 3B.

FIG. 3D shows quantification of results from H&E staining and IHC analysis for Ki67 and Caspase 3 expression in tumor tissue cultured in the tumor microenvironment platform treated with Pembrolizumab, Nivolumab, or IgG control for 3 days (T3) compared to baseline tumor tissue at TO for 2 additional patients (patient IDs 2918 and 2928).

FIG. 4 shows results for H&E staining and IHC analysis for Ki67, Caspase 3, and CD8 expression in tumor tissue cultured in the tumor microenvironment platform treated with Pembrolizumab, Nivolumab, or IgG control for 3 days (T3) compared to baseline tumor tissue at TO for patient ID 2941.

FIG. 5 shows FACS analysis for expression of CD3 and CD8 in cells from tumor tissue cultured in the tumor microenvironment platform treated with Pembrolizumab, Nivolumab, or IgG control for 3 days (T3) for patient IDs 2941 and 2942.

FIG. 6 shows results for IHC analysis for CD8 expression in tumor tissue cultured in the tumor microenvironment platform treated with Pembrolizumab, Nivolumab, or IgG control for 3 days (T3). Comparisons include control vs Nivo, control vs Pembro, Nivo vs Pembro, and control vs Nivo vs Pembro. Each line represents results from tumor tissue cultures prepared with tumor tissue from a single individual.

FIGS. 7A and 7B show results for IHC analysis for PD-1, FOXP3, and CD8 expression in tumor tissue cultured in the tumor microenvironment platform treated with Pembrolizumab, Nivolumab, or IgG control for 3 days (T3) compared to baseline tumor tissue at TO. FIG. 7A shows results for tumor tissue derived from a predicted responder to Pembrolizumab or Nivolumab. FIG. 7B shows results for tumor tissue derived from a predicted non-responder to Pembrolizumab or Nivolumab.

FIG. 8 shows results for IHC analysis for PD-L1⁺ tumor cells, PD-1⁺ T cells, and FOXP3⁺ T-regulatory cells in tumor tissue cultured in the tumor microenvironment platform treated with Pembrolizumab, Nivolumab, or IgG control for 3 days (T3). Comparisons include control vs Nivo, control vs Pembro, Nivo vs Pembro, and control vs Nivo vs Pembro. Each line represents results from tumor tissue cultures prepared with tumor tissue from a single individual.

FIGS. 9A and 9B show quantification of results for Granzyme B and Perforin secretion assays for HNSCC tumor tissue cultured in the tumor microenvironment platform treated with Pembrolizumab, Nivolumab, or IgG control for 24 or 48 hours. FIG. 9A shows results for tumor tissue derived from a predicted responder to Pembrolizumab or Nivolumab. FIG. 9B shows results for tumor tissue derived from a predicted non-responder to Pembrolizumab or Nivolumab.

FIGS. 10A and 10B show quantification of results for Granzyme B and Perforin secretion assays for CRC tumor tissue cultured in the tumor microenvironment platform treated with Ipilimumab, Nivolumab, Ipilimumab+Nivolumab, FOLFIRI, or IgG control for 24 or 48 hours. FIG. 10A shows results for tumor tissue derived from a predicted responder to Pembrolizumab or Nivolumab. FIG. 10B shows results for tumor tissue derived from a predicted non-responder to Pembrolizumab or Nivolumab.

### DETAILED DESCRIPTION

The present invention is based at least in part on the surprising discovery that a live human tumor tissue assay, optionally combined with a machine learning strategy, can accurately predict whether immune-modulatory agents (e.g., PD1 checkpoint inhibitors) will induce antitumor outcomes, and associated clinical response in an individual patient. Furthermore, it has been determined that in some cases this live tissue assay can detect differential antitumor responses to multiple drugs that target the same immune-modulatory protein in an individual patient (e.g., two distinct PD-1 checkpoint inhibitors, Nivolumab and Pembrolizumab). Described in this invention are specific phenotypic markers induced under therapy pressure which may be used to provide a quantitative measure of clinical outcome, for example, when being appropriately weighted by a machine learning algorithm. Accordingly, the present invention provides compositions, kits, articles of manufacture, and methods for predicting responsiveness of an individual having cancer to a therapeutic agent, such as an immunotherapeutic agent, including predicting differential responsiveness to agents targeting the same protein. Also provided are methods of treating cancer utilizing such predictive methods.

We have previously established and optimized a tumor microenvironment platform for culturing tumor tissue explants that mimics the native human tumor environment (see US Patent No. 2014/0228246, incorporated herein in its entirety). While this live tumor assay had been shown to accurately predict the antitumor effects of certain therapies, including small molecule kinase inhibitors, cytotoxic agents, and biological compound targeting oncogenes, it had yet to be demonstrated to predict the clinical outcome of immune modulatory agents such as immune check point inhibitors, one of a relatively new class of immune-oncology drugs that modulate the human immune system to target cancer cells. The present invention describes the use of a live tissue assay, which in some cases harnesses a multi-dimensional phenotypic “reflex” and optionally a machine learning algorithm, to predict the clinical outcome of cancer therapy drugs, such as immune modulatory drugs, in a single patient.

In some embodiments, the live tissue assay comprises a tumor tissue derived from an individual, an ECM composition, and optionally serum, plasma, peripheral blood nuclear cells (PBNCs), and/or granulocytes (such as autologous serum, plasma, PBNCs, and/or granulocytes). In some embodiments, the live tissue assay mimics aspects of the immune complex and compartment of the native tumor environment.

It is contemplated that in some embodiments, the live tumor tissue assay can accurately predict the clinical efficacy of a wide array of cancer therapeutic agents, including immunomodulatory agents. In some embodiments, the live tumor tissue assay is capable of accurately predicting differential clinical outcomes for related agents, such as cancer therapeutic agents targeting the same protein or pathway, or sharing a mechanism of action. It is also contemplated that in some embodiments, the invention can further predict the clinical efficacy of alternative immune modulatory therapeutics such as antitumor vaccines, chimeric antigen receptor T-cells (CAR-T), cytokine invigoration or even viral/bacterial immune stimulation strategies, and can be applicable to many different drugs and regimens including combination therapies.

### Definitions

Unless defined otherwise, the meanings of all technical and scientific terms used herein are those commonly understood by one of skill in the art to which this invention belongs. One of skill in the art will also appreciate that any methods and materials similar or equivalent to those described herein can also be used to practice or test the invention.

For use herein, unless clearly indicated otherwise, use of the terms “a”, “an,” and the like refers to one or more.

In this application, the use of “or” means “and/or” unless expressly stated or understood by one skilled in the art. In the context of a multiple dependent claim, the use of “or” refers back to more than one preceding independent or dependent claim.

Reference to “about” a value or parameter herein includes (and describes) embodiments that are directed to that value or parameter per se. For example, description referring to “about X” includes description of “X.”

It is understood that aspect and embodiments of the invention described herein include “comprising,” “consisting,” and “consisting essentially of” aspects and embodiments.

### Methods

### Predicting Responsiveness

In some embodiments, there is provided a method of predicting responsiveness to an immunotherapeutic agent (such as an immunomodulatory agent, e.g., an immune checkpoint inhibitor) for treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) converting the readout into a sensitivity index; and c) using the sensitivity index to predict responsiveness to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the immunotherapeutic agent is an immune checkpoint inhibitor. In some embodiments, the immune checkpoint inhibitor is an antagonist (e.g., antagonistic antibody) targeting an inhibitory immune checkpoint molecule. In some embodiments, the inhibitory immune checkpoint molecule is selected from CTLA4, PD-1, PD-L1, PD-L2, LAG3, B7-1, B7-H3, B7-H4, BTLA, VISTA, KIR, A2aR, and TIM3. In some embodiments, the immunotherapeutic agent is an agonist (e.g., agonistic antibody) targeting a stimulatory immune molecule. In some embodiments, the stimulatory immune molecule is selected from CD27, CD28, CD40, CD122, CD137, OX40, GITR, 4-1BB, HVEM, and ICOS. In some embodiments, the immunotherapeutic agent is pembrolizumab or nivolumab.

As used herein, a “readout” refers to a set of one or more assessment scores.

In some embodiments, according to any of the methods described herein employing a tumor microenvironment platform, the tumor microenvironment platform comprises an extracellular matrix composition. In some embodiments, the extracellular matrix composition comprises at least 2 (such as at least 3, 4, 5, or more) of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the extracellular matrix composition comprises no more than 6 (such as no more than 5, 4, 3, or fewer) of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the extracellular matrix composition comprises at least 2 (such as at least 3, 4, 5, or more) proteins selected from basement membrane proteins, cytoskeletal proteins, and matrix proteins. In some embodiments, the extracellular matrix composition comprises no more than 6 (such as no more than 5, 4, 3, or fewer) proteins selected from basement membrane proteins, cytoskeletal proteins, and matrix proteins. In some embodiments, the tumor microenvironment platform further comprises serum, plasma, and/or PBNCs. In some embodiments, at least one of the serum, plasma, and/or PBNCs are autologous to the individual. In some embodiments, at least one of the serum, plasma, and/or PBNCs are heterologous to the individual. In some embodiments, the PBNCs are peripheral blood mononuclear cells (PBMCs).

Thus, in some embodiments, according to any of the methods described herein employing a tumor microenvironment platform, the tumor microenvironment platform comprises a) an extracellular matrix composition comprising at least 2 (such as at least 3, 4, 5, or more) of collagen 1, collagen 3, collagen 4, collagen 6, Fibronectin, Vitronectin, Cadherin, Filamin A, Vimentin, Osteopontin, Laminin, Decorin, and Tenascin C; and b) serum, plasma, and/or PBNCs. In some embodiments, the extracellular matrix composition comprises no more than 6 (such as no more than 5, 4, 3, or fewer) of collagen 1, collagen 3, collagen 4, collagen 6, Fibronectin, Vitronectin, Cadherin, Filamin A, Vimentin, Osteopontin, Laminin, Decorin, and Tenascin C. In some embodiments, at least one of the serum, plasma, and/or PBNCs are autologous to the individual. In some embodiments, at least one of the serum, plasma, and/or PBNCs are heterologous to the individual. In some embodiments, the PBNCs are peripheral blood mononuclear cells (PBMCs).

In some embodiments, according to any of the methods described herein employing a tumor microenvironment platform, the tumor microenvironment platform comprises a) an extracellular matrix composition comprising at least 2 (such as at least 3, 4, 5, or more) proteins selected from basement membrane proteins, cytoskeletal proteins, and matrix proteins; and b) serum, plasma, and/or PBNCs. In some embodiments, the extracellular matrix composition comprises no more than 6 (such as no more than 5, 4, 3, or fewer) proteins selected from basement membrane proteins, cytoskeletal proteins, and matrix proteins. In some embodiments, at least one of the serum, plasma, and/or PBNCs are autologous to the individual. In some embodiments, at least one of the serum, plasma, and/or PBNCs are heterologous to the individual. In some embodiments, the PBNCs are peripheral blood mononuclear cells (PBMCs).

In some embodiments, according to any of the methods described herein employing an assessment score for an assay, the assessment score is generated based on a comparison between i) the result of the assay conducted on the tumor tissue culture treated with an agent (e.g., immunotherapeutic agent); and ii) the result of the assay conducted on a reference tumor tissue culture, wherein the reference tumor tissue culture comprises a tumor tissue from the individual cultured on the tumor microenvironment platform. In some embodiments, the assessment score is generated, for example, by taking the ratio of i) a numeric quantification of the result of the assay conducted on the tumor tissue culture treated with the agent to ii) the numeric quantification of the result of the assay conducted on the reference tumor tissue culture. In some embodiments, the reference tumor tissue culture is not treated with the agent.

In some embodiments, according to any of the methods described herein employing a tumor tissue culture from an individual, the method comprises culturing a tumor tissue from the individual on a tumor microenvironment platform as described herein to produce the tumor tissue culture.

In some embodiments, according to any of the methods described herein employing a plurality of assays conducted on a tumor tissue culture, the method comprises conducting the plurality of assays on the tumor tissue culture.

In some embodiments, there is provided a method of predicting responsiveness to an immunotherapeutic agent (such as an immunomodulatory agent, e.g., an immune checkpoint inhibitor) for treating cancer in an individual in need thereof, the method comprising: a) conducting a plurality of assays on a tumor tissue culture treated with the immunotherapeutic agent, wherein the tumor tissue culture comprises a tumor tissue from the individual cultured on a tumor microenvironment platform, and obtaining a readout comprising assessment scores from the plurality of assays; b) converting the readout into a sensitivity index; and c) using the sensitivity index to predict responsiveness to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the immunotherapeutic agent is an immune checkpoint inhibitor. In some embodiments, the immune checkpoint inhibitor is an antagonist (e.g., antagonistic antibody) targeting an inhibitory immune checkpoint molecule. In some embodiments, the inhibitory immune checkpoint molecule is selected from CTLA4, PD-1, PD-L1, PD-L2, LAG3, B7-1, B7-H3, B7-H4, BTLA, VISTA, KIR, A2aR, and TIM3. In some embodiments, the immunotherapeutic agent is an agonist (e.g., agonistic antibody) targeting a stimulatory immune molecule. In some embodiments, the stimulatory immune molecule is selected from CD27, CD28, CD40, CD122, CD137, OX40, GITR, 4-1BB, HVEM, and ICOS. In some embodiments, the immunotherapeutic agent is pembrolizumab or nivolumab.

In some embodiments, there is provided a method of predicting responsiveness to an immunotherapeutic agent (such as an immunomodulatory agent, e.g., an immune checkpoint inhibitor) for treating cancer in an individual in need thereof, the method comprising: a) preparing a tumor tissue culture by culturing a tumor tissue from the individual on a tumor microenvironment platform; b) conducting a plurality of assays on the tumor tissue culture that has been treated with the immunotherapeutic agent and obtaining a readout comprising assessment scores from the plurality of assays; c) converting the readout into a sensitivity index; and d) using the sensitivity index to predict responsiveness to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the immunotherapeutic agent is an immune checkpoint inhibitor. In some embodiments, the immune checkpoint inhibitor is an antagonist (e.g., antagonistic antibody) targeting an inhibitory immune checkpoint molecule. In some embodiments, the inhibitory immune checkpoint molecule is selected from CTLA4, PD-1, PD-L1, PD-L2, LAG3, B7-1, B7-H3, B7-H4, BTLA, VISTA, KIR, A2aR, and TIM3. In some embodiments, the immunotherapeutic agent is an agonist (e.g., agonistic antibody) targeting a stimulatory immune molecule. In some embodiments, the stimulatory immune molecule is selected from CD27, CD28, CD40, CD122, CD137, OX40, GITR, 4-1BB, HVEM, and ICOS. In some embodiments, the immunotherapeutic agent is pembrolizumab or nivolumab.

In some embodiments, there is provided a method of predicting responsiveness to an immunotherapeutic agent selected from pembrolizumab and nivolumab for treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) converting the readout into a sensitivity index; and c) using the sensitivity index to predict responsiveness to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of predicting responsiveness to an immunotherapeutic agent selected from pembrolizumab and nivolumab for treating cancer in an individual in need thereof, the method comprising: a) conducting a plurality of assays on a tumor tissue culture treated with the immunotherapeutic agent, wherein the tumor tissue culture comprises a tumor tissue from the individual cultured on a tumor microenvironment platform, and obtaining a readout comprising assessment scores from the plurality of assays; b) converting the readout into a sensitivity index; and c) using the sensitivity index to predict responsiveness to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of predicting responsiveness to an immunotherapeutic agent selected from pembrolizumab and nivolumab for treating cancer in an individual in need thereof, the method comprising: a) preparing a tumor tissue culture by culturing a tumor tissue from the individual on a tumor microenvironment platform; b) conducting a plurality of assays on the tumor tissue culture that has been treated with the immunotherapeutic agent and obtaining a readout comprising assessment scores from the plurality of assays; c) converting the readout into a sensitivity index; and d) using the sensitivity index to predict responsiveness to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of selecting a preferred therapeutic agent for treating cancer in an individual in need thereof from among a plurality of therapeutic agents against the same target molecule, the method comprising: a) obtaining a readout comprising an assessment score for each of a plurality of assays conducted on tumor tissue cultures treated individually with each of the plurality of therapeutic agents, wherein the tumor tissue culture comprises a tumor tissue from the individual cultured on a tumor microenvironment platform; b) converting the readout into sensitivity indices for each of the plurality of therapeutic agents; c) using the sensitivity indices to predict responsiveness of the individual to each of the plurality of therapeutic agents; and d) selecting from among the plurality of therapeutic agents the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different epitope on the target molecule. In some embodiments, at least some of the plurality of therapeutic agents target the same epitope on the target molecule. In some embodiments, the plurality of therapeutic agents are antibodies targeting the same epitope on the target molecule, wherein the antibodies have different sequences from each other. In some embodiments, the antibodies have different constant region sequences. In some embodiments, the antibodies have different variable region sequences. In some embodiments, the target molecule is a target protein. In some embodiments, the plurality of therapeutic agents comprise (such as consist of) pembrolizumab and nivolumab.

In some embodiments, there is provided a method of selecting a preferred therapeutic agent for treating cancer in an individual in need thereof from among a plurality of therapeutic agents against the same target molecule, the method comprising: a) conducting a plurality of assays on tumor tissue cultures treated individually with each of the plurality of therapeutic agents, wherein the tumor tissue culture comprises a tumor tissue from the individual cultured on a tumor microenvironment platform, and obtaining a readout comprising assessment scores from the plurality of assays; b) converting the readout into sensitivity indices for each of the plurality of therapeutic agents; c) using the sensitivity indices to predict responsiveness of the individual to each of the plurality of therapeutic agents; and d) selecting from among the plurality of therapeutic agents the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different epitope on the target molecule. In some embodiments, at least some of the plurality of therapeutic agents target the same epitope on the target molecule. In some embodiments, the plurality of therapeutic agents are antibodies targeting the same epitope on the target molecule, wherein the antibodies have different sequences from each other. In some embodiments, the antibodies have different constant region sequences. In some embodiments, the antibodies have different variable region sequences. In some embodiments, the target molecule is a target protein. In some embodiments, the plurality of therapeutic agents comprise (such as consist of) pembrolizumab and nivolumab.

In some embodiments, there is provided a method of selecting a preferred therapeutic agent for treating cancer in an individual in need thereof from among a plurality of therapeutic agents against the same target molecule, the method comprising: a) preparing a tumor tissue culture by culturing a tumor tissue from the individual on a tumor microenvironment platform; b) conducting a plurality of assays on the tumor tissue cultures that have been treated individually with each of the plurality of therapeutic agents; c) converting the readout into sensitivity indices for each of the plurality of therapeutic agents; d) using the sensitivity indices to predict responsiveness of the individual to each of the plurality of therapeutic agents; and e) selecting from among the plurality of therapeutic agents the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each have the same target molecule. In some embodiments, the plurality of therapeutic agents each target a different epitope on the target molecule. In some embodiments, at least some of the plurality of therapeutic agents target the same epitope on the target molecule. In some embodiments, the plurality of therapeutic agents are antibodies targeting the same epitope on the target molecule, wherein the antibodies have different sequences from each other. In some embodiments, the antibodies have different constant region sequences. In some embodiments, the antibodies have different variable region sequences. In some embodiments, the target molecule is a target protein. In some embodiments, the plurality of therapeutic agents comprise (such as consist of) pembrolizumab and nivolumab.

In some embodiments, there is provided a method of selecting a preferred therapeutic agent for treating cancer in an individual in need thereof from among a plurality of therapeutic agents targeting the same pathway, the method comprising: a) obtaining a readout comprising an assessment score for each of a plurality of assays conducted on tumor tissue cultures treated individually with each of the plurality of therapeutic agents, wherein the tumor tissue culture comprises a tumor tissue from the individual cultured on a tumor microenvironment platform; b) converting the readout into sensitivity indices for each of the plurality of therapeutic agents; c) using the sensitivity indices to predict responsiveness of the individual to each of the plurality of therapeutic agents; and d) selecting from among the plurality of therapeutic agents the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different protein in the pathway. In some embodiments, the plurality of therapeutic agents each target a different protein from a plurality of target proteins, and each of the plurality of target proteins directly target, or are a direct target of, another of the plurality of target proteins. In some embodiments, each of the plurality of therapeutic agents has a stimulatory effect on the pathway. In some embodiments, each of the plurality of therapeutic agents has an inhibitory effect on the pathway.

In some embodiments, there is provided a method of selecting a preferred therapeutic agent for treating cancer in an individual in need thereof from among a plurality of therapeutic agents against the same pathway, the method comprising: a) conducting a plurality of assays on tumor tissue cultures treated individually with each of the plurality of therapeutic agents, wherein the tumor tissue culture comprises a tumor tissue from the individual cultured on a tumor microenvironment platform, and obtaining a readout comprising assessment scores from the plurality of assays; b) converting the readout into sensitivity indices for each of the plurality of therapeutic agents; c) using the sensitivity indices to predict responsiveness of the individual to each of the plurality of therapeutic agents; and d) selecting from among the plurality of therapeutic agents the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different protein in the pathway. In some embodiments, the plurality of therapeutic agents each target a different protein from a plurality of target proteins, and each of the plurality of target proteins directly target, or are a direct target of, another of the plurality of target proteins. In some embodiments, each of the plurality of therapeutic agents has a stimulatory effect on the pathway. In some embodiments, each of the plurality of therapeutic agents has an inhibitory effect on the pathway.

In some embodiments, there is provided a method of selecting a preferred therapeutic agent for treating cancer in an individual in need thereof from among a plurality of therapeutic agents against the same pathway, the method comprising: a) preparing a tumor tissue culture by culturing a tumor tissue from the individual on a tumor microenvironment platform; b) conducting a plurality of assays on the tumor tissue cultures that have been treated individually with each of the plurality of therapeutic agents; c) converting the readout into sensitivity indices for each of the plurality of therapeutic agents; d) using the sensitivity indices to predict responsiveness of the individual to each of the plurality of therapeutic agents; and e) selecting from among the plurality of therapeutic agents the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different protein in the pathway. In some embodiments, the plurality of therapeutic agents each target a different protein from a plurality of target proteins, and each of the plurality of target proteins directly target, or are a direct target of, another of the plurality of target proteins. In some embodiments, each of the plurality of therapeutic agents has a stimulatory effect on the pathway. In some embodiments, each of the plurality of therapeutic agents has an inhibitory effect on the pathway.

In some embodiments, there is provided a method of selecting a preferred therapeutic agent for treating cancer in an individual in need thereof from among pembrolizumab and nivolumab, the method comprising: a) obtaining a readout comprising an assessment score for each of a plurality of assays conducted on tumor tissue cultures treated individually with pembrolizumab and nivolumab, wherein the tumor tissue culture comprises a tumor tissue from the individual cultured on a tumor microenvironment platform; b) converting the readout into sensitivity indices for pembrolizumab and nivolumab; c) using the sensitivity indices to predict responsiveness of the individual to pembrolizumab and nivolumab; and d) selecting from among pembrolizumab and nivolumab the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of selecting a preferred therapeutic agent for treating cancer in an individual in need thereof from among pembrolizumab and nivolumab, the method comprising: a) conducting a plurality of assays on tumor tissue cultures treated individually with pembrolizumab and nivolumab, wherein the tumor tissue culture comprises a tumor tissue from the individual cultured on a tumor microenvironment platform, and obtaining a readout comprising assessment scores from the plurality of assays; b) converting the readout into sensitivity indices for each of pembrolizumab and nivolumab; c) using the sensitivity indices to predict responsiveness of the individual to pembrolizumab and nivolumab; and d) selecting from among pembrolizumab and nivolumab the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of selecting a preferred therapeutic agent for treating cancer in an individual in need thereof from among pembrolizumab and nivolumab, the method comprising: a) preparing a tumor tissue culture by culturing a tumor tissue from the individual on a tumor microenvironment platform; b) conducting a plurality of assays on the tumor tissue cultures that have been treated individually with pembrolizumab and nivolumab; c) converting the readout into sensitivity indices for each of pembrolizumab and nivolumab; d) using the sensitivity indices to predict responsiveness of the individual to pembrolizumab and nivolumab; and e) selecting from among pembrolizumab and nivolumab the therapeutic agent with the highest sensitivity index as the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of predicting responsiveness to an immunotherapeutic agent for treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; and d) using the output to predict responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a method of classifying likely responsiveness to an immunotherapeutic agent for treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; and d) using the output to classify the likely responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output classifies complete clinical response, partial clinical response, or no clinical response of the individual to administration of the immunotherapeutic agent. In some embodiments, the output classifies response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a computer-implemented method for predicting responsiveness to an immunotherapeutic agent for treating cancer in an individual in need thereof, the method comprising: a) accessing a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; and d) using the output to predict responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a non-transitory computer-readable storage medium storing computer executable instructions that when executed by a computer control the computer to perform a method for predicting responsiveness to an immunotherapeutic agent for treating cancer in an individual in need thereof, the method comprising: a) accessing a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) receiving, from the predictive model, an output; and d) using the output to predict responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a system for generating a report of the predicted responsiveness to an immunotherapeutic agent for treating cancer in an individual in need thereof comprising: a) at least one computer database comprising: a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; and b) a computer-readable program code comprising instructions to: i) input the readout into a predictive model; ii) receive, from the predictive model, an output; iii) use the output to predict responsiveness of the individual to administration of the immunotherapeutic agent; and iv) generate a report that comprises the predicted responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a method of selecting a preferred therapeutic agent for treating cancer in an individual in need thereof from among a plurality of therapeutic agents against the same target molecule, the method comprising: a) obtaining a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform; b) inputting the readout into a predictive model; c) using the predictive model to generate an output for each of the plurality of therapeutic agents; d) using the outputs to predict responsiveness of the individual to administration of each of the plurality of therapeutic agents, and e) selecting from among the plurality of therapeutic agents the therapeutic agent with the highest predicted responsiveness as the preferred therapeutic agent. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

In some embodiments, there is provided a method of selecting a preferred therapeutic agent for treating cancer in an individual in need thereof from among a plurality of therapeutic agents against the same target molecule, the method comprising: a) obtaining a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform; b) inputting the readout into a predictive model; c) using the predictive model to generate an output for each of the plurality of therapeutic agents; d) using the outputs to classify responsiveness of the individual to administration of each of the plurality of therapeutic agents, and e) selecting from among the plurality of therapeutic agents the therapeutic agent with the highest classified responsiveness as the preferred therapeutic agent. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent classifies complete clinical response, partial clinical response, or no clinical response of the individual to administration of the given therapeutic agent. In some embodiments, the output for a given therapeutic agent classifies response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

In some embodiments, there is provided a computer-implemented method of selecting a preferred therapeutic agent for treating cancer in an individual in need thereof from among a plurality of therapeutic agents against the same target molecule, the method comprising: a) accessing a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform; b) inputting the readout into a predictive model; c) using the predictive model to generate an output for each of the plurality of therapeutic agents; d) using the outputs to predict responsiveness of the individual to administration of each of the plurality of therapeutic agents, and e) selecting from among the plurality of therapeutic agents the therapeutic agent with the highest predicted responsiveness as the preferred therapeutic agent. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

In some embodiments, there is provided a non-transitory computer-readable storage medium storing computer executable instructions that when executed by a computer control the computer to perform a method for selecting a preferred therapeutic agent for treating cancer in an individual in need thereof from among a plurality of therapeutic agents against the same target molecule, the method comprising: a) accessing a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform; b) inputting the readout into a predictive model; c) receiving, from the predictive model, an output for each of the plurality of therapeutic agents; d) using the outputs to predict responsiveness of the individual to administration of each of the plurality of therapeutic agents, and e) selecting from among the plurality of therapeutic agents the therapeutic agent with the highest predicted responsiveness as the preferred therapeutic agent. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

In some embodiments, there is provided a system for generating a report of a preferred therapeutic agent for treating cancer in an individual in need thereof from among a plurality of therapeutic agents against the same target molecule comprising: a) at least one computer database comprising: a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform; and b) a computer-readable program code comprising instructions to: i) input the readout into a predictive model; ii) receive, from the predictive model, an output for each of the plurality of therapeutic agents; iii) use the outputs to predict responsiveness of the individual to administration of each of the plurality of therapeutic agents; iv) select from among the plurality of therapeutic agents the therapeutic agent with the highest predicted responsiveness as the preferred therapeutic agent; and v) generate a report that comprises the preferred therapeutic agent. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

In some embodiments, there is provided an assay method comprising a) conducting a plurality of assays on a tumor tissue culture treated with an immunotherapeutic agent, wherein the tumor tissue culture comprises a tumor tissue from an individual cultured on a tumor microenvironment platform; and b) generating a readout comprising an assessment score for each of the plurality of assays, wherein the readout is used to predict responsiveness of the individual to administration of the immunotherapeutic agent. In some embodiments, using the readout to predict responsiveness of the individual to administration of the immunotherapeutic agent comprises c) inputting the readout into a predictive model; d) using the predictive model to generate an output; and e) using the output to predict responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises preparing the tumor tissue culture by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided an assay method comprising a) conducting a plurality of assays on tumor tissue cultures treated individually with each of a plurality of therapeutic agents against the same target molecule, wherein the tumor tissue cultures each comprises a tumor tissue from the individual cultured on a tumor microenvironment platform; and b) generating a readout comprising an assessment score for each of the plurality of assays, wherein the readout is used to predict responsiveness of the individual to administration of each of the plurality of therapeutic agents, and wherein the therapeutic agent with the highest predicted responsiveness from among the plurality of therapeutic agents is selected as a preferred therapeutic agent. In some embodiments, using the readout to predict responsiveness of the individual to administration of each of the plurality of therapeutic agents comprises c) inputting the readout into a predictive model; d) using the predictive model to generate an output for each of the plurality of therapeutic agents; and e) using the outputs to predict responsiveness of the individual to administration of each of the plurality of therapeutic agents. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

It is also contemplated that any of the methods described herein can be used for predicting the responsiveness to a combination of immunotherapeutic agents for treating cancer in an individual in need thereof. In some such embodiments, the immunotherapeutic agent of the method is replaced with a combination of immunotherapeutic agents. Treatment of tissue culture with a combination of immunotherapeutic agents is well known in the art, and any such methods of treatment can be used in any of the methods described herein. For example, in some embodiments, each of the combination of immunotherapeutic agents is added to the tissue culture simultaneously. In some embodiments, at least some of the combination of immunotherapeutic agents are added to the tissue culture at different times, such as sequentially or concurrently.

Treatment

In some embodiments, there is provided a method of treating cancer in an individual in need thereof, the method comprising: a) obtaining a readout comprising an assessment score for each of a plurality of assays conducted on a tumor tissue culture treated with an immunotherapeutic agent, wherein the tumor tissue culture comprises a tumor tissue from the individual cultured on a tumor microenvironment platform; b) converting the readout into a sensitivity index; c) using the sensitivity index to predict responsiveness to the immunotherapeutic agent; and d) administering the immunotherapeutic agent to the individual if the individual is predicted to respond to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the immunotherapeutic agent is an immune checkpoint inhibitor. In some embodiments, the immune checkpoint inhibitor is an antagonist (e.g., antagonistic antibody) targeting an inhibitory immune checkpoint molecule. In some embodiments, the inhibitory immune checkpoint molecule is selected from CTLA4, PD-1, PD-L1, PD-L2, LAG3, B7-1, B7-H3, B7-H4, BTLA, VISTA, KIR, A2aR, and TIM3. In some embodiments, the immunotherapeutic agent is an agonist (e.g., agonistic antibody) targeting a stimulatory immune molecule. In some embodiments, the stimulatory immune molecule is selected from CD27, CD28, CD40, CD122, CD137, OX40, GITR, 4-1BB, HVEM, and ICOS. In some embodiments, the immunotherapeutic agent is pembrolizumab or nivolumab.

In some embodiments, there is provided a method of treating cancer in an individual in need thereof, the method comprising: a) conducting a plurality of assays on a tumor tissue culture treated with an immunotherapeutic agent, wherein the tumor tissue culture comprises a tumor tissue from the individual cultured on a tumor microenvironment platform, and obtaining a readout comprising assessment scores from the plurality of assays; b) converting the readout into a sensitivity index; c) using the sensitivity index to predict responsiveness to the immunotherapeutic agent; and d) administering the immunotherapeutic agent to the individual if the individual is predicted to respond to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the immunotherapeutic agent is an immune checkpoint inhibitor. In some embodiments, the immune checkpoint inhibitor is an antagonist (e.g., antagonistic antibody) targeting an inhibitory immune checkpoint molecule. In some embodiments, the inhibitory immune checkpoint molecule is selected from CTLA4, PD-1, PD-L1, PD-L2, LAG3, B7-1, B7-H3, B7-H4, BTLA, VISTA, KIR, A2aR, and TIM3. In some embodiments, the immunotherapeutic agent is an agonist (e.g., agonistic antibody) targeting a stimulatory immune molecule. In some embodiments, the stimulatory immune molecule is selected from CD27, CD28, CD40, CD122, CD137, OX40, GITR, 4-1BB, HVEM, and ICOS. In some embodiments, the immunotherapeutic agent is pembrolizumab or nivolumab.

In some embodiments, there is provided a method of treating cancer in an individual in need thereof, the method comprising: a) preparing a tumor tissue culture by culturing a tumor tissue from the individual on a tumor microenvironment platform; b) conducting a plurality of assays on the tumor tissue culture that has been treated with an immunotherapeutic agent and obtaining a readout comprising assessment scores from the plurality of assays; c) converting the readout into a sensitivity index; d) using the sensitivity index to predict responsiveness to the immunotherapeutic agent; and e) administering the immunotherapeutic agent to the individual if the individual is predicted to respond to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the immunotherapeutic agent is an immune checkpoint inhibitor. In some embodiments, the immune checkpoint inhibitor is an antagonist (e.g., antagonistic antibody) targeting an inhibitory immune checkpoint molecule. In some embodiments, the inhibitory immune checkpoint molecule is selected from CTLA4, PD-1, PD-L1, PD-L2, LAG3, B7-1, B7-H3, B7-H4, BTLA, VISTA, KIR, A2aR, and TIM3. In some embodiments, the immunotherapeutic agent is an agonist (e.g., agonistic antibody) targeting a stimulatory immune molecule. In some embodiments, the stimulatory immune molecule is selected from CD27, CD28, CD40, CD122, CD137, OX40, GITR, 4-1BB, HVEM, and ICOS. In some embodiments, the immunotherapeutic agent is pembrolizumab or nivolumab.

In some embodiments, there is provided a method of treating cancer in an individual in need thereof, the method comprising: a) obtaining a readout comprising an assessment score for each of a plurality of assays conducted on a tumor tissue culture treated with an immunotherapeutic agent selected from pembrolizumab and nivolumab, wherein the tumor tissue culture comprises a tumor tissue from the individual cultured on a tumor microenvironment platform; b) converting the readout into a sensitivity index; c) using the sensitivity index to predict responsiveness to the immunotherapeutic agent; and d) administering the immunotherapeutic agent to the individual if the individual is predicted to respond to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of treating cancer in an individual in need thereof, the method comprising: a) conducting a plurality of assays on a tumor tissue culture treated with an immunotherapeutic agent selected from pembrolizumab and nivolumab, wherein the tumor tissue culture comprises a tumor tissue from the individual cultured on a tumor microenvironment platform, and obtaining a readout comprising assessment scores from the plurality of assays; b) converting the readout into a sensitivity index; c) using the sensitivity index to predict responsiveness to the immunotherapeutic agent; and d) administering the immunotherapeutic agent to the individual if the individual is predicted to respond to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of treating cancer in an individual in need thereof, the method comprising: a) preparing a tumor tissue culture by culturing a tumor tissue from the individual on a tumor microenvironment platform; b) conducting a plurality of assays on the tumor tissue culture that has been treated with an immunotherapeutic agent selected from pembrolizumab and nivolumab and obtaining a readout comprising assessment scores from the plurality of assays; c) converting the readout into a sensitivity index; d) using the sensitivity index to predict responsiveness to the immunotherapeutic agent; and e) administering the immunotherapeutic agent to the individual if the individual is predicted to respond to the immunotherapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of treating cancer in an individual in need thereof, the method comprising: a) obtaining a readout comprising an assessment score for each of a plurality of assays conducted on tumor tissue cultures treated individually with each of a plurality of therapeutic agents against the same target molecule, wherein the tumor tissue culture comprises a tumor tissue from the individual cultured on a tumor microenvironment platform; b) converting the readout into sensitivity indices for each of the plurality of therapeutic agents; c) using the sensitivity indices to predict responsiveness of the individual to each of the plurality of therapeutic agents; d) selecting from among the plurality of therapeutic agents the therapeutic agent with the highest sensitivity index as the preferred therapeutic agent; and e) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different epitope on the target molecule. In some embodiments, at least some of the plurality of therapeutic agents target the same epitope on the target molecule. In some embodiments, the plurality of therapeutic agents are antibodies targeting the same epitope on the target molecule, wherein the antibodies have different sequences from each other. In some embodiments, the antibodies have different constant region sequences. In some embodiments, the antibodies have different variable region sequences. In some embodiments, the target molecule is a target protein. In some embodiments, the plurality of therapeutic agents comprise (such as consist of) pembrolizumab and nivolumab.

In some embodiments, there is provided a method of treating cancer in an individual in need thereof, the method comprising: a) conducting a plurality of assays on tumor tissue cultures treated individually with each of a plurality of therapeutic agents against the same target molecule, wherein the tumor tissue culture comprises a tumor tissue from the individual cultured on a tumor microenvironment platform, and obtaining a readout comprising assessment scores from the plurality of assays; b) converting the readout into sensitivity indices for each of the plurality of therapeutic agents; c) using the sensitivity indices to predict responsiveness of the individual to each of the plurality of therapeutic agents; d) selecting from among the plurality of therapeutic agents the therapeutic agent with the highest sensitivity index as the preferred therapeutic agent; and e) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different epitope on the target molecule. In some embodiments, at least some of the plurality of therapeutic agents target the same epitope on the target molecule. In some embodiments, the plurality of therapeutic agents are antibodies targeting the same epitope on the target molecule, wherein the antibodies have different sequences from each other. In some embodiments, the antibodies have different constant region sequences. In some embodiments, the antibodies have different variable region sequences. In some embodiments, the target molecule is a target protein. In some embodiments, the plurality of therapeutic agents comprise (such as consist of) pembrolizumab and nivolumab.

In some embodiments, there is provided a method of treating cancer in an individual in need thereof, the method comprising: a) preparing a tumor tissue culture by culturing a tumor tissue from the individual on a tumor microenvironment platform; b) conducting a plurality of assays on the tumor tissue cultures that have been treated individually with each of a plurality of therapeutic agents against the same target molecule; c) converting the readout into sensitivity indices for each of the plurality of therapeutic agents; d) using the sensitivity indices to predict responsiveness of the individual to each of the plurality of therapeutic agents; e) selecting from among the plurality of therapeutic agents the therapeutic agent with the highest sensitivity index as the preferred therapeutic agent; and f) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each have the same target molecule. In some embodiments, the plurality of therapeutic agents each target a different epitope on the target molecule. In some embodiments, at least some of the plurality of therapeutic agents target the same epitope on the target molecule. In some embodiments, the plurality of therapeutic agents are antibodies targeting the same epitope on the target molecule, wherein the antibodies have different sequences from each other. In some embodiments, the antibodies have different constant region sequences. In some embodiments, the antibodies have different variable region sequences. In some embodiments, the target molecule is a target protein. In some embodiments, the plurality of therapeutic agents comprise (such as consist of) pembrolizumab and nivolumab.

In some embodiments, there is provided a method of treating cancer in an individual in need thereof, the method comprising: a) obtaining a readout comprising an assessment score for each of a plurality of assays conducted on tumor tissue cultures treated individually with each of a plurality of therapeutic agents targeting the same pathway, wherein the tumor tissue culture comprises a tumor tissue from the individual cultured on a tumor microenvironment platform; b) converting the readout into sensitivity indices for each of the plurality of therapeutic agents; c) using the sensitivity indices to predict responsiveness of the individual to each of the plurality of therapeutic agents; d) selecting from among the plurality of therapeutic agents the therapeutic agent with the highest sensitivity index as the preferred therapeutic agent; and e) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different protein in the pathway. In some embodiments, the plurality of therapeutic agents each target a different protein from a plurality of target proteins, and each of the plurality of target proteins directly target, or are a direct target of, another of the plurality of target proteins. In some embodiments, each of the plurality of therapeutic agents has a stimulatory effect on the pathway. In some embodiments, each of the plurality of therapeutic agents has an inhibitory effect on the pathway.

In some embodiments, there is provided a method of treating cancer in an individual in need thereof, the method comprising: a) conducting a plurality of assays on tumor tissue cultures treated individually with each of a plurality of therapeutic agents against the same pathway, wherein the tumor tissue culture comprises a tumor tissue from the individual cultured on a tumor microenvironment platform, and obtaining a readout comprising assessment scores from the plurality of assays; b) converting the readout into sensitivity indices for each of the plurality of therapeutic agents; c) using the sensitivity indices to predict responsiveness of the individual to each of the plurality of therapeutic agents; d) selecting from among the plurality of therapeutic agents the therapeutic agent with the highest sensitivity index as the preferred therapeutic agent; and e) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different protein in the pathway. In some embodiments, the plurality of therapeutic agents each target a different protein from a plurality of target proteins, and each of the plurality of target proteins directly target, or are a direct target of, another of the plurality of target proteins. In some embodiments, each of the plurality of therapeutic agents has a stimulatory effect on the pathway. In some embodiments, each of the plurality of therapeutic agents has an inhibitory effect on the pathway.

In some embodiments, there is provided a method of treating cancer in an individual in need thereof, the method comprising: a) preparing a tumor tissue culture by culturing a tumor tissue from the individual on a tumor microenvironment platform; b) conducting a plurality of assays on the tumor tissue cultures that have been treated individually with each of a plurality of therapeutic agents against the same pathway; c) converting the readout into sensitivity indices for each of the plurality of therapeutic agents; d) using the sensitivity indices to predict responsiveness of the individual to each of the plurality of therapeutic agents; e) selecting from among the plurality of therapeutic agents the therapeutic agent with the highest sensitivity index as the preferred therapeutic agent; and f) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response. In some embodiments, the plurality of therapeutic agents comprise a chemotherapeutic agent, such as a cytostatic or cytotoxic agent. In some embodiments, the plurality of therapeutic agents comprise a targeted therapeutic agent, such as a targeted antibody or targeted small molecule (e.g., protein inhibitor, such as kinase inhibitor). In some embodiments, the plurality of therapeutic agents comprise an immunotherapeutic agent, such as an immunomodulatory agent, e.g., an immune checkpoint inhibitor (such as an antagonistic antibody targeting an immune checkpoint molecule) or an immunostimulatory agent (such as an agonistic antibody targeting an immunostimulatory molecule). In some embodiments, the plurality of therapeutic agents are antibodies. In some embodiments, the plurality of therapeutic agents each target a different protein in the pathway. In some embodiments, the plurality of therapeutic agents each target a different protein from a plurality of target proteins, and each of the plurality of target proteins directly target, or are a direct target of, another of the plurality of target proteins. In some embodiments, each of the plurality of therapeutic agents has a stimulatory effect on the pathway. In some embodiments, each of the plurality of therapeutic agents has an inhibitory effect on the pathway.

In some embodiments, there is provided a method of treating cancer in an individual in need thereof, the method comprising: a) obtaining a readout comprising an assessment score for each of a plurality of assays conducted on tumor tissue cultures treated individually with pembrolizumab and nivolumab, wherein the tumor tissue culture comprises a tumor tissue from the individual cultured on a tumor microenvironment platform; b) converting the readout into sensitivity indices for pembrolizumab and nivolumab; c) using the sensitivity indices to predict responsiveness of the individual to pembrolizumab and nivolumab; d) selecting from among pembrolizumab and nivolumab the therapeutic agent with the highest sensitivity index as a preferred therapeutic agent; and e) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of treating cancer in an individual in need thereof, the method comprising: a) conducting a plurality of assays on tumor tissue cultures treated individually with pembrolizumab and nivolumab, wherein the tumor tissue culture comprises a tumor tissue from the individual cultured on a tumor microenvironment platform, and obtaining a readout comprising assessment scores from the plurality of assays; b) converting the readout into sensitivity indices for each of pembrolizumab and nivolumab; c) using the sensitivity indices to predict responsiveness of the individual to pembrolizumab and nivolumab; d) selecting from among pembrolizumab and nivolumab the therapeutic agent with the highest sensitivity index as the preferred therapeutic agent; and e) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of treating cancer in an individual in need thereof, the method comprising: a) preparing a tumor tissue culture by culturing a tumor tissue from the individual on a tumor microenvironment platform; b) conducting a plurality of assays on the tumor tissue cultures that have been treated individually with pembrolizumab and nivolumab; c) converting the readout into sensitivity indices for each of pembrolizumab and nivolumab; d) using the sensitivity indices to predict responsiveness of the individual to pembrolizumab and nivolumab; e) selecting from among pembrolizumab and nivolumab the therapeutic agent with the highest sensitivity index as the preferred therapeutic agent; and f) administering the preferred therapeutic agent to the individual if the individual is predicted to respond to the preferred therapeutic agent. In some embodiments, the tumor microenvironment platform comprises an extracellular matrix composition comprising one or more of (such as at least 3, 4, 5, or more of) collagen 1, collagen 3, collagen 4, collagen 6, Fibronectin, Vitronectin, Cadherin, Filamin A, Vimentin, Osteopontin, Laminin, Decorin, Tenascin C, Basement membrane proteins, Cytoskeletal proteins and Matrix proteins. In some embodiments, the tumor microenvironment platform further comprises serum, plasma, and/or PBNCs. In some embodiments, the serum, plasma, and/or PBNCs are autologous to the individual. In some embodiments, the serum, plasma, and/or PBNCs are heterologous to the individual. In some embodiments, the plurality of assays comprise one or more assays selected from cell viability assays, cell death assays, cell proliferation assays, tumor morphology assays, tumor stroma content assays, cell metabolism assays, senescence assays, cytokine profile assays, enzyme activity assays, and tumor and/or stromal cell expression assays. In some embodiments, converting the readout into a sensitivity index comprises using a predictive model (such as a machine-trained predictive model) with weightage coefficients for each of the plurality of assays to obtain weighted assessment scores for each of the plurality of assays, and combining the weighted assessment scores to yield the sensitivity index. In some embodiments, the predictive model comprises as an output one of a plurality of degrees of responsiveness, each of which is associated with a different range of non-overlapping values, and using the sensitivity index to predict responsiveness comprises predicting the responsiveness to be the degree of responsiveness associated with the range of values in which the sensitivity index lies. In some embodiments, the plurality of degrees of responsiveness comprises (such as consists of) clinical response and no clinical response. In some embodiments, the plurality of degrees of responsiveness comprises (such as consists of) complete clinical response, partial clinical response, and no clinical response.

In some embodiments, there is provided a method of treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; d) using the output to predict responsiveness of the individual to administration of the immunotherapeutic agent; and e) administering the immunotherapeutic agent to the individual if the individual is predicted to respond to the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a method of treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) inputting the readout into a predictive model; c) using the predictive model to generate an output; d) using the output to classify responsiveness of the individual to administration of the immunotherapeutic agent; and e) administering the immunotherapeutic agent to the individual if the individual is classified to respond to the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output classifies complete clinical response, partial clinical response, or no clinical response of the individual to administration of the immunotherapeutic agent. In some embodiments, the output classifies response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a method of treating cancer in an individual in need thereof, the method comprising 1) using a non-transitory computer-readable storage medium storing computer executable instructions that when executed by a computer control the computer to: a) obtain a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; b) input the readout into a predictive model; c) use the predictive model to generate an output; and d) use the output to predict responsiveness of the individual to administration of the immunotherapeutic agent; and 2) administering the immunotherapeutic agent to the individual if the individual is predicted to respond to the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a method of treating cancer in an individual in need thereof, the method comprising 1) using a system for generating a report of the predicted responsiveness of the individual to administration of an immunotherapeutic agent comprising: a) at least one computer database comprising: a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform; and b) a computer-readable program code comprising instructions to: i) input the readout into a predictive model; ii) receive, from the predictive model, an output; iii) use the output to predict responsiveness of the individual to administration of the immunotherapeutic agent; and iv) generate a report that comprises the predicted responsiveness of the individual to administration of the immunotherapeutic agent; and 2) administering the immunotherapeutic agent to the individual if the individual is predicted to respond to the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided a method of treating cancer in an individual in need thereof, the method comprising: a) obtaining a readout comprising an assessment score for each of a plurality of assays conducted on tumor tissue cultures treated individually with each of a plurality of therapeutic agents against the same target molecule, wherein the tumor tissue cultures each comprises a tumor tissue from the individual cultured on a tumor microenvironment platform; b) inputting the readout into a predictive model; c) using the predictive model to generate an output for each of the plurality of therapeutic agents; d) using the outputs to predict responsiveness of the individual to administration of each of the plurality of therapeutic agents, e) selecting from among the plurality of therapeutic agents the therapeutic agent with the highest predicted responsiveness as the preferred therapeutic agent; and f) administering the preferred therapeutic agent to the individual if the individual is predicted to respond to the preferred therapeutic agent. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

In some embodiments, there is provided an assay method comprising a) conducting a plurality of assays on a tumor tissue culture treated with an immunotherapeutic agent, wherein the tumor tissue culture comprises a tumor tissue from an individual cultured on a tumor microenvironment platform; and b) generating a readout comprising an assessment score for each of the plurality of assays, wherein the readout is used to predict responsiveness of the individual to administration of the immunotherapeutic agent, and wherein the immunotherapeutic agent is adminsitered to the individual if the individual is predicted to respond to the immunotherapeutic agent. In some embodiments, using the readout to predict responsiveness of the individual to administration of the immunotherapeutic agent comprises c) inputting the readout into a predictive model; d) using the predictive model to generate an output; and e) using the output to predict responsiveness of the individual to administration of the immunotherapeutic agent. In some embodiments, the predictive model comprises an algorithm that uses each of the assessment scores as input and generates the output. In some embodiments, the algorithm comprises multiplying each of the input assessment scores with a corresponding weightage coefficient to obtain a plurality of weighted assessment scores; and combining the plurality of weighted assessment scores to generate the output. In some embodiments, the output predicts complete clinical response, partial clinical response, or no clinical response of the individual to administration of the immunotherapeutic agent. In some embodiments, the output predicts response or no response of the individual to administration of the immunotherapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises preparing the tumor tissue culture by culturing tumor tissue from the individual in the tumor microenvironment platform. In some embodiments, the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with the immunotherapeutic agent. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the immunotherapeutic agent is an immune checkpoint inhibitor. In some embodiments, the immune checkpoint inhibitor is an antagonistic antibody targeting an immune checkpoint molecule. In some embodiments, the immune checkpoint inhibitor is pembrolizumab or nivolumab. In some embodiments, the individual is human.

In some embodiments, there is provided an assay method comprising a) conducting a plurality of assays on tumor tissue cultures treated individually with each of a plurality of therapeutic agents against the same target molecule, wherein the tumor tissue cultures each comprises a tumor tissue from the individual cultured on a tumor microenvironment platform; and b) generating a readout comprising an assessment score for each of the plurality of assays, wherein the readout is used to predict responsiveness of the individual to administration of each of the plurality of therapeutic agents, wherein the therapeutic agent with the highest predicted responsiveness from among the plurality of therapeutic agents is selected as a preferred therapeutic agent, and wherein the preferred therapeutic agent is administered to the individual if the individual is predicted to respond to the preferred therapeutic agent. In some embodiments, using the readout to predict responsiveness of the individual to administration of each of the plurality of therapeutic agents comprises c) inputting the readout into a predictive model; d) using the predictive model to generate an output for each of the plurality of therapeutic agents; and e) using the outputs to predict responsiveness of the individual to administration of each of the plurality of therapeutic agents. In some embodiments, the predictive model comprises an algorithm that, for each of the plurality of therapeutic agents, uses each of the assessment scores for the given therapeutic agent as input and generates the output for the given therapeutic agent. In some embodiments, the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent. In some embodiments, the output for a given therapeutic agent predicts complete clinical response, partial clinical response, or no clinical response of the individual to administration of the given therapeutic agent. In some embodiments, the output for a given therapeutic agent predicts response or no response of the individual to administration of the given therapeutic agent. In some embodiments, the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C. In some embodiments, the tumor microenvironment platform further comprises serum, plasma, and/or peripheral blood nuclear cells (PBNCs). In some embodiments, one or more of the serum, plasma, and/or PBNCs are derived from the individual. In some embodiments, step a) further comprises preparing the tumor tissue cultures by culturing tumor tissue from the individual in the tumor microenvironment platform. In some embodiments, the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform. In some embodiments, the reference tumor tissue culture is not treated with any of the plurality of therapeutic agents. In some embodiments, step a) further comprises conducting the plurality of assays on the reference tumor tissue culture; and/or step a) further comprises preparing the reference tumor tissue culture by culturing tumor tissue from the individual on the tumor microenvironment platform. In some embodiments, the plurality of therapeutic agents comprises a plurality of immune checkpoint inhibitors. In some embodiments, the plurality of immune checkpoint inhibitors comprises a plurality of antagonistic antibodies targeting an immune checkpoint molecule. In some embodiments, the plurality of immune checkpoint inhibitors comprises pembrolizumab and nivolumab. In some embodiments, the individual is human.

It is also contemplated that any of the methods described herein can be used for treating cancer in an individual in need thereof by predicting the responsiveness of the individual to a combination of therapeutic agents. In some such embodiments, the therapeutic agent of the method is replaced with a combination of therapeutic agents. Treatment of tissue culture with a combination of therapeutic agents is well known in the art, and any such methods of treatment can be used in any of the methods described herein. For example, in some embodiments, each of the combination of therapeutic agents is added to the tissue culture simultaneously. In some embodiments, at least some of the combination of therapeutic agents are added to the tissue culture at different times, such as sequentially or concurrently.

In some embodiments, according to any of the methods described herein, the individual is human.

### Tumor Microenvironment Platform

The methods described herein in some embodiments employ a tumor microenvironment platform for culturing tumor tissue, said microenvironment comprising an Extra Cellular Matrix (ECM) composition and culture medium, and optionally including serum, plasma, and/or peripheral blood nuclear cells (PBNCs), such as peripheral blood mononuclear cells (PBMCs). In some embodiments, the tumor microenvironment platform further comprises one or more immune factors. In some embodiments, the tumor microenvironment platform further comprises one or more angiogenic factors. In some embodiments, the tumor microenvironment platform further comprises one or more drugs, such as one or more cancer therapeutic agents (e.g., immunomodulatory agents, such as immune checkpoint inhibitors).

In some embodiments, the serum, plasma, and/or PBNCs are derived from an individual according to any of the methods described herein. For example, according to a method of predicting responsiveness to a therapeutic agent for treating cancer in an individual in need thereof described herein, the serum, plasma, and/or PBNCs are derived from the individual (i.e., autologous). In some embodiments, the serum, plasma, and/or PBNCs are not derived from the individual (i.e., heterologous). In some embodiments, the serum and/or plasma is xenogeneic.

In some embodiments, the one or more immune factors are isolated from serum or plasma derived from an individual according to any of the methods described herein (i.e., autologous serum or plasma). In some embodiments, the one or more immune factors are isolated from serum or plasma not derived from the individual (i.e., heterologous serum or plasma). In some embodiments, the serum or plasma is xenogeneic.

In some embodiments, the one or more angiogenic factors are isolated from serum or plasma derived from an individual according to any of the methods described herein (i.e., autologous serum or plasma). In some embodiments, the one or more angiogenic factors are isolated from serum or plasma not derived from the individual (i.e., heterologous serum or plasma). In some embodiments, the serum or plasma is xenogeneic.

In some embodiments, the ECM composition comprises at least three components selected from group consisting of collagen 1, collagen 3, collagen 4, collagen 6, Fibronectin, Vitronectin, Cadherin, Filamin A, Vimentin, Laminin, Decorin, Tenascin C, Osteopontin, Basement membrane proteins, Cytoskeletal proteins and Matrix proteins.

In some embodiments, the components of the ECM composition are specific to tissue from a tumor, and are selected by subjecting a sample of the tumor tissue to one or more assays to identify components of the ECM present in the tumor tissue (e.g., mass spectrometry, such as liquid chromatography-mass spectrometry (LCMS)), and selecting from among the identified ECM components at least three components selected from the group consisting of collagen 1, collagen 3, collagen 4, collagen 6, Fibronectin, Vitronectin, Cadherin, Filamin A, Vimentin, Laminin, Decorin, Tenascin C, Osteopontin, Basement membrane proteins, Cytoskeletal proteins and Matrix proteins. In some embodiments, the tumor is, for example, a stomach, colon, head & neck, brain, oral cavity, breast, gastric, gastro-intestinal, oesophageal, colorectal, pancreatic, lung (e.g., non-small cell lung or small cell lung), liver, kidney, ovarian, uterine, bone, prostate, testicular, thyroid, or bladder tumor. In some embodiments, the tumor is a glioblastoma, astrocytoma, or melanoma. Also contemplated are ECM compositions specific for hematological cancers including AML (Acute Myeloid Leukemia), CML (Chronic Myelogenous Leukemia), ALL (Acute Lymphocytic Leukemia), TALL (T-cell Acute Lymphoblastic Leukemia), NHL (Non-Hodgkins Lymphoma), DBCL (Diffuse B-cell Lymphoma), CLL (Chronic Lymphocytic Leukemia) and multiple myeloma. In some embodiments, the ECM composition comprises ECM components identified from a sample of bone marrow. In some embodiments, the ECM composition comprises ECM components identified from a sample of blood plasma. In some embodiments, the ECM composition comprises ECM components identified from an autologous sample (e.g., the tumor tissue in the tumor microenvironment platform is derived from the same individual as the sample from which the ECM components are identified). In some embodiments, the ECM composition comprises ECM components identified from a heterologous sample (e.g., the tumor tissue in the tumor microenvironment platform is derived from a different individual than the sample from which the ECM components are identified).

In some embodiments, the ECM composition comprises collagen 1 at a concentration ranging from about 0.01 μg/ml to about 100 μg/ml, such as at about 5 μg/ml or about 20 μg/ml or about 50 μg/ml. In some embodiments, the ECM composition comprises collagen 3 at a concentration ranging from about 0.01 μg/ml to about 100 μg/ml, such as at about 0.1 μg/ml or about 1 μg/ml or about 100 μg/ml. In some embodiments, the ECM composition comprises collagen 4 at a concentration ranging from about 0.01 μg/ml to about 500 μg/ml, such as at about 5 μg/ml or about 20 μg/ml or about 250 μg/ml. In some embodiments, the ECM composition comprises collagen 6 at a concentration ranging from about 0.01 μg/ml to about 500 μg/ml, such as at about 0.1 μg/ml or about 1 μg/ml or about 10 μg/ml. In some embodiments, the ECM composition comprises Fibronectin at a concentration ranging from about 0.01 μg/ml to about 750 μg/ml, such as at about 5 μg/ml or about 20 μg/ml or about 500 μg/ml. In some embodiments, the ECM composition comprises Vitronectin at a concentration ranging from about 0.01 μg/ml to about 95 μg/ml, such as at about 5 μg/ml or about 10 μg/ml. In some embodiments, the ECM composition comprises Cadherin at a concentration ranging from about 0.01 μg/ml to about 500 μg/ml, such as at about 1 μg/ml and about 5 μg/ml. In some embodiments, the ECM composition comprises Filamin A at a concentration ranging from about 0.01 μg/ml to about 500 μg/ml, such as at about 5 μg/ml or about 10 μg/ml. In some embodiments, the ECM composition comprises Vimentin at a concentration ranging from about 0.01 μg/ml to about 100 μg/ml, such as at about 1 μg/ml or about 10 μg/ml. In some embodiments, the ECM composition comprises Laminin at a concentration ranging from about 0.01 μg/ml to about 100 μg/ml, such as at about 5 μg/ml or about 10 μg/ml or about 20 μg/ml. In some embodiments, the ECM composition comprises Decorin at concentration ranging from about 0.01 μg/ml to about 100 μg/ml, such as at about 10 μg/ml or about 20 μg/ml. In some embodiments, the ECM composition comprises Tenascin C at a concentration ranging from about 0.01 μg/ml to about 500 μg/ml, such as at about 10 μg/ml or about 25 μg/ml. In some embodiments, the ECM composition comprises Osteopontin at a concentration ranging from about 0.01 μg/ml to about 150 μg/ml, such as at about 1 μg/ml or about 5 μg/ml. In some embodiments, the ECM composition comprises one or more Basement membrane proteins at a concentration ranging from about 0.01 μg/ml to about 150 μg/ml. In some embodiments, the ECM composition comprises one or more cytoskeletal proteins at a concentration ranging from about 0.01 μg/ml to about 150 μg/ml. In some embodiments, the ECM composition comprises one or more matrix proteins at a concentration ranging from about 0.01 μg/ml to about 150 μg/ml.

In some embodiments, the tumor microenvironment platform comprises a substrate coated with the ECM composition. In some embodiments, the substrate is, for example, a plate, base, flask, dish, petriplate, or petridish. The substrate may be made of any material suitable for being coated with the ECM composition. In some embodiments, the substrate is coated with the EMC composition by depositing a liquid mixture comprising the ECM composition on the substrate and allowing the liquid mixture to dry. In some embodiments, the liquid mixture is an aqueous mixture. In some embodiments, the liquid mixture is allowed to dry at a temperature at least about 25 (such as at least about any of 25, 26, 27, 28, 29, 30, 31, 32, 33, 34, 35, 36, 37, 38, 39, 40, 41, 42, or more, including any ranges between these values) ° C. In some embodiments, the substrate is washed with an appropriate solution (e.g., a buffer, such as PBS) at least 1× (such as at least 1×, 2×, 3×, or more) following coating with the ECM composition. In some embodiments, the substrate has been stored at a temperature no greater than about 4 (such as no greater than about any of 4, 0, −5, −10, −15, −20, −25, −30, or less, including any ranges between these values) ° C. prior to combination with culture medium.

In some embodiments, the culture medium is combined with the ECM composition by overlaying the culture medium on a substrate coated with the ECM composition. In some embodiments, the culture medium comprises Dulbecco's Modified Eagle Medium (DMEM) or RPMI1640 (Roswell Park Memorial Institute Medium), for example DMEM or RPMI1640 at a concentration ranging from about 60% to about 100%, such as about 80%. In some embodiments, the culture medium comprises serum, such as heat inactivated FBS (Foetal Bovine Serum), for example FBS at a concentration ranging from about 0.1% to about 40%, such as about 2% wt/wt. In some embodiments, the serum is added to the culture medium after culturing the tumor tissue in the culture medium for a duration of time. In some embodiments, the serum is added to the culture medium after culturing the tumor tissue in the culture medium for at least 6 hours (such as at least about any of 6, 7, 8, 9, 10, 11, 12, 14, 16, 18, 20, 22, or 24 hours or more). In some embodiments, the culture medium comprises Penicillin-Streptomycin at a concentration ranging from about 1% to about 2%, such as about 1% wt/wt. In some embodiments, the culture medium comprises sodium pyruvate at a concentration ranging from about 10 mM to about 500 mM, such as about 100 mM. In some embodiments, the culture medium comprises a nonessential amino acid, including, but not limited to, L-glutamine, at a concentration ranging from about 1 mM to about 10 mM, such as about 5 mM. In some embodiments, the culture medium comprises HEPES ((4-(2-hydroxyethyl)-1-piperazineethanesulfonic acid) at concentration ranging from about 1 mM to about 20 mM, preferably about 10 mM; the serum, is at concentration ranging from about 0.1% to about 10%, preferably about 2%. In some embodiments, the culture medium is exchanged at regular intervals. In some embodiments, the culture medium is exchanged at an interval of at least about 12 hours (such as at least about any of 12, 14, 16, 18, 20, 22, 24, 30, 36, 40, 44, 48, 60, or 72 hours or more).

In some embodiments, the one or more drugs are present in the culture medium before it is combined with the ECM composition. In some embodiments, at least one of the one or more drugs is added to the culture medium after it is combined with the ECM composition. In some embodiments, each of the one or more drugs is added to the culture medium after it is combined with the ECM composition. In some embodiments, at least some of the one or more drugs are added to the culture medium at different times. For example, in some embodiments, at least one of the one or more drugs is added to the culture medium before it is combined with the ECM compositions, and at least one of the one or more drugs is added to the culture medium after it is combined with the ECM composition. In some embodiments, at least some of the one or more drugs are added to the culture medium at different times after it is combined with the ECM composition. In some embodiments, at least some of the one or more drugs are cancer therapeutic agents. In some embodiments, each of the one or more drugs are cancer therapeutic agents. In some embodiments, the one or more drugs comprise a chemotherapeutic agent, such as a cytostatic or cytotoxic agent. In some embodiments, the one or more drugs comprise a targeted cancer therapeutic agent, such as a targeted antibody or targeted small molecule drug (e.g., protein inhibitor, such as kinase inhibitor). In some embodiments, the one or more drugs comprise an immunomodulatory agent, such as an immune checkpoint inhibitor or immunostimulatory agent. In some embodiments, the one or more drugs comprise one or more agents selected from alkylating agents, anthracycline agents, antibodies, cytoskeletal disrupting agents (e.g., taxanes), epothilones, histone deacetylase inhibitors (HDACi), kinase inhibitors, macrolides, nucleotide analogs and precursor analogs, peptide antibiotics, platinum-based agents, retinoids, topoisomerase inhibitors (e.g., topoisomerase I or topoisomerase II inhibitors), and vinca alkaloids and derivatives.

The term “immunomodulatory agent” refers to a therapeutic agent that when present, alters, suppresses or stimulates the body's immune system. Immunomodulators can include compositions or formulations that activate the immune system (e.g., adjuvants or activators), or downregulate the immune system. Adjuvants can include aluminum-based compositions, as well as compositions that include bacterial or mycobacterial cell wall components. Activators can include molecules that activate antigen presenting cells to stimulate the cellular immune response. For example, activators can be immunostimulant peptides. Activators can include, but are not limited to, agonists of toll-like receptors TLR-2, 3, 4, 6, 7, 8, or 9, granulocyte macrophage colony stimulating factor (GM-CSF); TNF; CD40L; CD28; FLT-3 ligand; or cytokines such as IL-1, IL-2, IL-4, IL-7, IL-12, IL-15, or IL-21. Activators can include agonists of activating receptors (including co-stimulatory receptors) on T cells, such as an agonist (e.g., agonistic antibody) of CD28, OX40, ICOS, GITR, 4-1BB, CD27, CD40, or HVEM. Activators can also include compounds that inhibit the activity of an immune suppressor, such as an inhibitor of the immune suppressors IL-10, IL-35, FasL, TGF-β, indoleamine-2,3 dioxygenase (IDO), or cyclophosphamide, or inhibit the activity of an immune checkpoint such as an antagonist (e.g., antagonistic antibody) of CTLA4, PD-1, PD-L1, PD-L2, LAG3, B7-1, B7-H3, B7-H4, BTLA, VISTA, KIR, A2aR, or TIM3. Activators can also include costimulatory molecules such as CD40, CD80, or CD86. Immunomodulators can also include agents that downregulate the immune system such as antibodies against IL-12p70, antagonists of toll-like receptors TLR-2, 3, 4, 5, 6, 8, or 9, or general suppressors of immune function such as cyclophosphamide, cyclosporin A or FK506. Other antibodies of interest include those directed to tumor cell targets, including for example anti-CD38 antibody (such as daratumumab). These agents (e.g., adjuvants, activators, or downregulators) can be combined to shape an optimal immune response.

The term “immune checkpoint inhibitor” refers to compounds that inhibit the activity of control mechanisms of the immune system. Immune system checkpoints, or immune checkpoints, are inhibitory pathways in the immune system that generally act to maintain self-tolerance or modulate the duration and amplitude of physiological immune responses to minimize collateral tissue damage. Immune checkpoint inhibitors can inhibit an immune system checkpoint by inhibiting the activity of a protein in the pathway. Immune system checkpoint proteins include, but are not limited to, cytotoxic T-lymphocyte antigen 4 (CTLA4), programmed cell death 1 protein (PD-1), programmed cell death 1 ligand 1 (PD-L1), programmed cell death 1 ligand 2 (PD-L2), lymphocyte activation gene 3 (LAG3), B7-1, B7-H3, B7-H4, T cell membrane protein 3 (TIM3), B- and T-lymphocyte attenuator (BTLA), V-domain immunoglobulin (Ig)-containing suppressor of T-cell activation (VISTA), Killer-cell immunoglobulin-like receptor (KIR), and A2A adenosine receptor (A2aR). As such, immune checkpoint inhibitors include antagonists of CTLA4, PD-1, PD-L1, PD-L2, LAG3, B7-1, B7-H3, B7-H4, BTLA, VISTA, KIR, A2aR, or TIM3. For example, antibodies that bind to CTLA4, PD-1, PD-L1, PD-L2, LAG3, B7-1, B7-H3, B7-H4, BTLA, VISTA, KIR, A2aR, or TIM3 and antagonize their function are checkpoint inhibitors. Moreover, any molecule (e.g., peptide, nucleic acid, small molecule, etc.) that inhibits the inhibitory function of an immune system checkpoint is an immune checkpoint inhibitor.

In some embodiments, according to any of the methods described herein, the immunomodulatory agent enhances an immune response in the individual and may include, but is not limited to, a cytokine, a chemokine, a stem cell growth factor, a lymphotoxin, an hematopoietic factor, a colony stimulating factor (CSF), erythropoietin, thrombopoietin, tumor necrosis factor-alpha (TNF), TNF-beta, granulocyte-colony stimulating factor (G-CSF), granulocyte macrophage-colony stimulating factor (GM-CSF), interferon-alpha, interferon-beta, interferon-gamma, interferon-lambda, stem cell growth factor designated “Si factor”, human growth hormone, N-methionyl human growth hormone, bovine growth hormone, parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, mullerian-inhibiting substance, mouse gonadotropin-associated peptide, inhibin, activin, vascular endothelial growth factor, integrin, NGF-beta, platelet-growth factor, TGF-alpha, TGF-beta, insulin-like growth factor-I, insulin-like growth factor-II, macrophage-CSF (M-CSF), IL-1, IL-la, IL-2, IL-3, IL-4, IL-5, IL-6, IL-7, IL-8, IL-9, IL-10, IL-11, IL-12, IL-13, IL-14, IL-15, IL-16, IL-17, IL-18, IL-21, IL-25, LIF, FLT-3, angiostatin, thrombospondin, endostatin, lymphotoxin, thalidomide, lenalidomide, or pomalidomide. In some embodiments, the immunomodulator is pomalidomide or an enantiomer or a mixture of enantiomers thereof, or a pharmaceutically acceptable salt, solvate, hydrate, co-crystal, clathrate, or polymorph thereof. In some embodiments, the immunomodulator is lenalidomide or an enantiomer or a mixture of enantiomers thereof, or a pharmaceutically acceptable salt, solvate, hydrate, co-crystal, clathrate, or polymorph thereof.

In some embodiments, according to any of the methods described herein, the immunomodulatory agent enhances an immune response in the individual and may include, but is not limited to, an antagonistic antibody selected from the group consisting of anti-CTLA4 (such as Ipilimumab and Tremelimumab), anti-PD-1 (such as Nivolumab, Pidilizumab, and Pembrolizumab), anti-PD-L1 (such as MPDL3280A, BMS-936559, MEDI4736, and Avelumab), anti-PD-L2, anti-LAG3 (such as BMS-986016 or C9B7W), anti-B7-1, anti-B7-H3 (such as MGA271), anti-B7-H4, anti-TIM3, anti-BTLA, anti-VISTA, anti-KIR (such as Lirilumab and IPH2101), anti-A2aR, anti-CD52 (such as alemtuzumab), anti-IL-10, anti-IL-35, anti-FasL, and anti-TGF-β (such as Fresolumimab). In some embodiments, the antibody is a monoclonal antibody. In some embodiments, the antibody is human or humanized.

In some embodiments, according to any of the methods described herein, the immunomodulator enhances an immune response in the individual and may include, but is not limited to, an agonistic antibody selected from the group consisting of anti-CD28, anti-OX40 (such as MEDI6469), anti-ICOS (such as JTX-2011, Jounce Therapeutics), anti-GITR (such as TRX518), anti-4-1BB (such as BMS-663513 and PF-05082566), anti-CD27 (such as Varlilumab and hCD27.15), anti-CD40 (such as CP870,893), and anti-HVEM. In some embodiments, the antibody is a monoclonal antibody. In some embodiments, the antibody is human or humanized

In some embodiments, the tumor tissue cultured in the tumor microenvironment platform is primary tumor tissue derived from an individual (e.g., a human), such as by standard protocols (e.g., by excision during surgery or by biopsy). In some embodiments, the tumor tissue cultured in the tumor microenvironment platform is from a tumor xenograft derived from primary tumor tissue from a first individual (e.g., a human) that has been implanted (e.g., subcutaneously) in a second individual (e.g., an immune-compromised mouse, such as a SCID mouse). In some embodiments, tumor tissue from a tumor xenograft is excised from the xenograft after it has reached a threshold volume. In some embodiments, the threshold volume is at least about 500 (such as at least about any of 500, 600, 700, 800, 900, 1000, 1200, 1400, 1600, 1800, 2000, or more, including any ranges between these values) mm³. Tumor tissue can be excised according to any of the methods of tumor excision known in the art. In some embodiments, the tumor tissue is a tissue section having a thickness from about 100 μm to about 3000 μm (such as about any of 100, 200, 300, 400, 500, 600, 700, 800, 900, 1000, 1200, 1400, 1600, 1800, 2000, 2200, 2400, 2600, 2800, or 3000 μm, including any ranges between these values).

In some embodiments, there is provided a method of producing a tumor microenvironment platform for culturing tumor tissue, the method comprising coating a substrate with an ECM composition according to any of the embodiments described herein and overlaying the coated substrate with culture medium, optionally along with serum, plasma and/or PBNC (such as autologous serum, plasma and/or PBNCs). In some embodiments, one or more drugs, such as cancer therapeutic agents (e.g., immunomodulatory agents, such as immune checkpoint inhibitors), are included in the culture medium. In some embodiments, the one or more drugs are included in the culture medium prior to overlaying the coated substrate. In some embodiments, the one or more drugs are added to the culture medium after overlaying the coated substrate.

In some embodiments, there is provided a method of organotypic culturing of a tumor tissue, the method comprising culturing the tumor tissue on a tumor microenvironment platform according to any of the embodiments described herein, thereby producing an organotypic culture.

In some embodiments, according to any of the methods described herein, the tumor tissue is obtained from a source selected from the group consisting of central nervous system, bone marrow, blood, spleen, thymus, heart, mammary gland, liver, pancreas, thyroid, skeletal muscle, kidney, lung, intestine, stomach, esophagus, ovary, bladder, testis, uterus, stromal tissue and connective tissue, or any combinations thereof. In some embodiments, the tumor tissue is obtained by excision during surgery or by biopsy (such as punch biopsy). In some embodiments, the tumor tissue is derived from a xenograft implant. In some embodiments, a section of the tumor tissue having a thickness of about 100 μm to about 3000 μm is used for culturing in the tumor microenvironment platform. In some embodiments, tumor tissue having a volume of about 0.2 cm³ to about 0.5 cm³ is used for culturing in the tumor microenvironment platform.

In some embodiments, according to any of the methods described herein, culturing of the tumor tissue is carried out at a temperature ranging from about 30° C. to about 40° C., such as at about 37° C. In some embodiments, culturing of the tumor tissue is carried out for a duration of time ranging from about 2 days to 10 days, such as about 3 days to 7 days. In some embodiments, culturing of the tumor tissue is carried out at about 5% CO₂.

### Readout Assays

In some embodiments, the plurality of assays used for producing the readout according to any of the methods described herein include both kinetic and end-point assays. In some embodiments, the plurality of assays include cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof. In some embodiments, the plurality of assays comprise (such as consist of) no more than 10 assays (such as no more than any of 9, 8, 7, 6, 5, 4, or 3 assays).

In some embodiments, the assays for cell viability include, for example, MTT assay, WST assay, ATP uptake assay and glucose uptake assay. In some embodiments, the assays for cell proliferation and metabolism include, for example, Ki67 assay, PCNA (proliferating nuclear cell antigen) assay, ATP/ADP ratio assay, and glucose uptake assay. In some embodiments, the assays for cell death include, for example, lactose dehydrogenase (LDH) assay, Activated Caspase 3 assay, Activated Caspase 8 assay, Nitric Oxide Synthase assay, and TUNEL assay. In some embodiments, the assays for senescence include, for example, senescence-associated beta-galactosidase staining. In some embodiments, the assays for tumor morphology and tumor stroma include, for example, Haemaotxylin & Eosin staining (H&E) for tumor cell content, size of the tumor cells, ratio of viable cells/dead cells, ratio of tumor cells/normal cells, tumor/macrophage ratio, nuclear size, density, and integrity, apoptotic bodies, and mitotic figures. In some embodiments, one or more of the plurality of assays is an immunohistochemical assay, including multi-plexed immunohistochemical assays, such as for evaluating simultaneous activity/infiltration of immune cells and/or signaling/activity components. In some embodiments, one or more of the plurality of assays is a quantitative or qualitative assay including, for example, ELISA, blotting (e.g., Western, Northern, or Southern blot), LC/MS, bead based assay, immune-depletion assay, and chromatographic assay. In some embodiments, one or more of the plurality of assays comprises a fluorogenic probe, such as a probe that generates a fluorescent signature following cleavage (e.g., enzymatic cleavage, such as by granzyme, caspase-1, TNFa-converting enzyme (TACE), or matrix metalloprotease) of a substrate.

In some embodiments, the cytokine profile assays include assays for one or more of TGF-0, IFN-γ, IL-6, GM-CSF, ILlb, IL-4, TNFa, IL-23/12, CD40/CD40L, and IL-8. In some embodiments, the cytokine profile assays include one or more immunohistochemical and/or flow cytometric assays for cells expressing the cytokines. In some embodiments, the cytokine profile assays include one or more cytokine secretion assays, such as ELISA-based assays for determining secretion of the cytokines.

In some embodiments, the enzyme activity assays include assays (such as ELISA-based assays) to determine the concentration of enzymes (such as secreted enzymes, e.g., granzyme) in the tumor tissue culture.

In some embodiments, the plurality of assays comprise assays (such as ELISA-based assays) to determine the concentration of cytolytic proteins (such as cytotoxic T cell cytolytic proteins, e.g., perforin) in the tumor tissue culture.

In some embodiments, each of the plurality of assays is assigned a numeric assessment score based on the results of the assay under treated and control conditions. The numeric assessment score can be based on any number of transformations of the assay results into a numeric representation, such as those used conventionally in the art for the particular assay. In some embodiments, the assessment score is determined as the fold change in a numeric output of the assay with treatment as compared to control. For example, in some embodiments, the assay is for determining the amount of a particular cell type (e.g., CD8+ T cell) in the tissue culture as a percent of total cells, with an output of 40% for the treated condition vs 20% for the control condition, and the assessment score is determined as 2, based on the two-fold increase. In some embodiments, the assessment score is determined based on the increase of a numeric output of the assay with treatment as compared to control. For example, in some embodiments, the assay is for determining the amount of a particular cell type (e.g., CD8+ T cell) in the tissue culture as a percent of total cells, with an output of 40% for the treated condition vs 20% for the control condition, and the assessment score is determined as 20, based on the 20% increase. In some embodiments, the assessment score is determined based on the percent inhibition of a numeric output of the assay with treatment as compared to control. For example, in some embodiments, the assay is a viability assay with 70% viability for treatment compared to control, and the assessment score is determined as 30, based on the 30% inhibition in viability. In some embodiments, the assessments scores are determined such that increasing values correspond to increasing degrees of response to treatment. For example, in some embodiments, the assay is a tumor cell viability assay with an assessment score based on an output of % inhibition in tumor cell viability for treatment compared to control, where 100% inhibition is more likely to predict a stronger response to treatment than 0% inhibition. In some embodiments, all of the assessment scores are determined such that they fall within the same predetermined range. For example, in some embodiments, all of the assessment score are determined such that they range between 0 and 100.

### Predictive Model

The methods described herein in some embodiments employ a predictive model used to generate an output for an individual based on assessment scores from assays conducted on tumor tissue explants derived from the individual cultured in a tumor microenvironment platform as described herein, and treated with a drug or combination of drugs. In some embodiments, the output predicts responsiveness of the individual to treatment with the drug or combination of drugs. In some embodiments, the output is used to classify the likely responsiveness of an individual to treatment with the drug or combination of drugs. In some embodiments, the output is a sensitivity index. The terms “sensitivity index” and “M-score” are used herein interchangeably. In some embodiments, the predictive model comprises weightage coefficients for each of the plurality of assays, and the output (e.g., sensitivity index) is generated by multiplying the numeric assessment score of each of the plurality of assays with its weightage score to obtain a weighted assessment score for each of the plurality of assays, and adding together each of the weighted assessment scores to obtain the output (e.g., sensitivity index).

In some embodiments, the weightage coefficients associated with each of the assays used for generating the output (e.g., sensitivity index) in the predictive model are determined using a machine learning algorithm. See Majumder, B., et al. Nature communications. 6, 2015, incorporated by reference herein in its entirety. In some embodiments, tumor tissue samples derived from a number of individuals prior to their treatment with a drug or combination of drugs are used to obtain results from a plurality of tumor tissue explant assays as described herein, which are transformed into numeric assessment scores, and the assessment scores for each individual paired with their associated clinical outcome (e.g., PERCIST/RECIST tumor response metrics, such as complete clinical response, partial clinical response, and no clinical response) following treatment are input into the machine learning algorithm, whereby the machine learning algorithm outputs weightage coefficients for each of the assays such that the sensitivity indices for the number of individual (calculated for each individual by multiplying their assessment score for each of the assays with its associated weightage score to generate weighted assessment scores, and adding together these weighted assessment scores) correlate (e.g., linearly correlate) with their clinical outcome. In some embodiments, the machine learning algorithm comprises multivariate analysis carried out on a computer to arrive at a predictive model with weightage coefficients for each of the assays that minimizes the deviation between the predicted clinical response and the observed clinical response for the number of individuals (i.e., maximizes the correlation between output (e.g., sensitivity index) and clinical outcome for the number of individuals). In some embodiments, the sensitivity indices have a positive predictive value (PPV) greater than at least about 80% (such as greater than at least about 81, 82, 83, 84, 85, 86, 87, 88, 89, 90, 91, 92, 93, 94, 95, 96, 97, 98, or 99%). In some embodiments, the sensitivity indices have a negative predictive value (NPV) greater than at least about 80% (such as greater than at least about 81, 82, 83, 84, 85, 86, 87, 88, 89, 90, 91, 92, 93, 94, 95, 96, 97, 98, or 99%). In some embodiments, clinical outcomes for the number of individuals are assessed after completion of at least 3 (such as at least 3, 4, 5, 6, or more) cycles of treatment. In some embodiments, the number of individuals is at least about 50 (such as at least about any of 60, 70, 80, 90, 100, 200, 300, 400, 500, 600, 700, 800, 900, 1000, 1250, 1500, 1750, 2000, or more, including any ranges between these values).

In some embodiments, the methods described herein employ a machine learning algorithm trained on a training set. In some embodiments, the training set comprises n examples (xᵢ,yᵢ), i=1, . . . , n, wherein xᵢ is a feature vector comprising m assessment scores for the i-th patient and yᵢ is a value corresponding to clinical response for the i-th patient (e.g., 1 if the i-th patient is a responder and −1 if the i-th patient is a non-responder). In some embodiments, the machine learning algorithm is trained on the training set such that the false positive rate is less than about 30% (such less than about any of 25, 20, 15, 10, 9, 8, 7, 6, 5, 4, 3, 2, or 1%). In some embodiments, the machine learning algorithm is trained on the training set in one stage. For example, in some embodiments, the machine learning algorithm is trained on the training set in one stage to predict response or non-response for new test cases. In some embodiments, the machine learning algorithm is trained on the training set in one stage to predict response or non-response for new test cases, wherein yᵢ is 1 if the i-th patient is a responder and −1 if the i-th patient is a non-responder. In some embodiments, the machine learning algorithm is trained on the training set in at least 2 (such as at least 3, 4, 5, or more) stages. For example, in some embodiments, the machine learning algorithm is trained on the training set in at least 2 (such as at least 3, 4, 5, or more) stages to predict non-response and 2 or more classes of response (e.g., complete response and partial response) for new test cases. For example, in some embodiments, the machine learning algorithm is trained on the training set in a first stage and a second stage to predict non-response, complete response, and partial response for new test cases, wherein the first stage comprises training the machine learning algorithm on the training set to generate an initial model for response/non-response, and wherein the second stage comprises further refining the initial model to classify the predicted responders as partial-responders or complete responders.

In some embodiments, the machine learning algorithm is the SVMpAUC algorithm (Narasimhan, N. & Agarwal, S. Proceedings of the 19th ACM SIGKDD Conference on Knowledge Discovery and Data Mining. 167-175, 2013). In some embodiments, the SVMpAUC algorithm is trained on a training set comprising n examples (xᵢ,yᵢ), i=1, . . . , n, wherein xᵢ is a feature vector containing m assessment scores for the i-th patient and yᵢ is 1 if the i-th patient is a responder and −1 otherwise. In some embodiments, the SVMpAUC algorithm learns a model comprising a weight vector w comprising weightage coefficients for each of the m assessment scores maximizing (a concave lower bound on) the partial area under the ROC curve (partial AUC) up to a specified false positive rate/3 (e.g., β=0.25), defined as

$$pAUC ( w ) = {\sum }_{i : {y}_{i} = 1} {\sum }_{j : {y}_{i} = - 1} 1 ( ω \cdot {x}_{i} > w \cdot {x}_{j} ) \cdot 1 ( j ∈ {S}_{β} ) ,$$

wherein S_{β} contains indices j of the top β fraction of non-responders in the training set, ranked according to scores w.xⱼ (Chu, W. & Keerthi, S. S. Neural Comput. 19, 792-815, 2007). In some embodiments, the model further comprises a first threshold value separating non-responders from responders in the training set with a false positive rate of about β. In some embodiments, the model further comprises a second threshold value separating partial responders from complete responders, wherein the second threshold value is selected to maximize the classification accuracy of the model for partial responders and complete responders on the training set.

In some embodiments, the possible numeric assessment scores and associated weightage coefficients for each of the assays included in the output (e.g., sensitivity index) generation for a predictive model are selected such that the output (e.g., sensitivity index) can range from a predetermined minimum to a predetermined maximum. In some embodiments, the minimum is 0 and the maximum is 100. In some embodiments, the output (e.g., sensitivity index) predicts varying degrees of responsiveness to one or more therapeutic agents in the individual. In some embodiments, the output (e.g., sensitivity index) predicts at least 2 (such as at least 2, 3, 4, 5, 6, or more) degrees of responsiveness to one or more therapeutic agents in the individual. In some embodiments, the output (e.g., sensitivity index) predicts clinical response or no clinical response to one or more therapeutic agents in the individual. In some embodiments, the output (e.g., sensitivity index) predicts complete clinical response, partial clinical response, or no clinical response to one or more therapeutic agents in the individual. In some embodiments, the output (e.g., sensitivity index) predicts complete clinical response, partial clinical response, no response, or no clinical response to one or more therapeutic agents in the individual. In some embodiments, the output (e.g., sensitivity index) is generated such that one or more threshold values separate ranges in the output (e.g., sensitivity index) that correlate with a degree of response to one or more therapeutic agents in the individual. In some embodiments, the output (e.g., sensitivity index) is generated such that a value above a threshold value predicts clinical response and a value below the threshold value predicts no clinical response in the individual. In some embodiments, the output (e.g., sensitivity index) is generated such that a value above an upper threshold value predicts complete clinical response, a value between the upper threshold value and a lower threshold value predicts partial clinical response, and a value below the lower threshold value predicts no clinical response in the individual. Such configurations can be adapted to accommodate prediction of any number of degrees of responsiveness. In some embodiments, the output (e.g., sensitivity index) range and the one or more threshold values are predetermined, such as to maximize ability to discriminate between degrees of clinical outcomes, and used as inputs in the machine learning algorithm for assigning weightage coefficients. For example, in some embodiments, a) the output (e.g., sensitivity index) can range from 0 to 100, and has an upper threshold value of 60 and a lower threshold value of 20; and b) the machine learning algorithm outputs weightage coefficients for each of the plurality of assays to maximize i) correlation of sensitivity indices ranging from 0-20 with no clinical response; ii) correlation of sensitivity indices ranging from 20-60 with partial clinical response; and iii) correlation of sensitivity indices ranging from 60-100 with complete clinical response. Various output (e.g., sensitivity index) ranges and numbers and values of thresholds are contemplated, and can be selected to suit any given purpose for predicting any number of degrees of responsiveness.

### EXAMPLES

The examples, which are intended to be purely exemplary of the invention and should therefore not be considered to limit the invention in any way, also describe and detail aspects and embodiments of the invention discussed above. The examples are not intended to represent that the experiments below are all or the only experiments performed.

### Example 1. A Patient-Derived Ex Vivo Tumor Microenvironment Platform Predicts Distinct Therapeutic Outcomes to Multiple PD-1 Checkpoint Inhibitors in Single Tumor Biopsies

Here, we employed a patient-derived ex-vivo tumor explant culture system based on a tumor microenvironment platform (see US Patent No. 2014/0228246), which serves to mimic the native 3D tumor microenvironment, autocrine-paracrine dynamic, and response to therapy by incorporating fresh tumor tissue and autologous immune cells with immunotherapy agents. Utilizing primary and late stage HNSCC patient samples (n=16) we interrogated phenotypic response to both Pembrolizumab (KEYTRUDA) and Nivolumab (OPDIVO), two FDA-approved PD-1 inhibitors, as single agents on the tumor microenvironment platform with tumor tissue from the same patient. To do this, we assayed tumor proliferation (Ki67) and apoptosis (active caspase-3), in addition to using a comprehensive panel of immunological assays to evaluate changes in the immune compartment (CD8, CD45, FOXP3, CXCR4, CD68, PDL1, PD1) and cytokine profile (IL6, IL8, IFN-g and IL12).

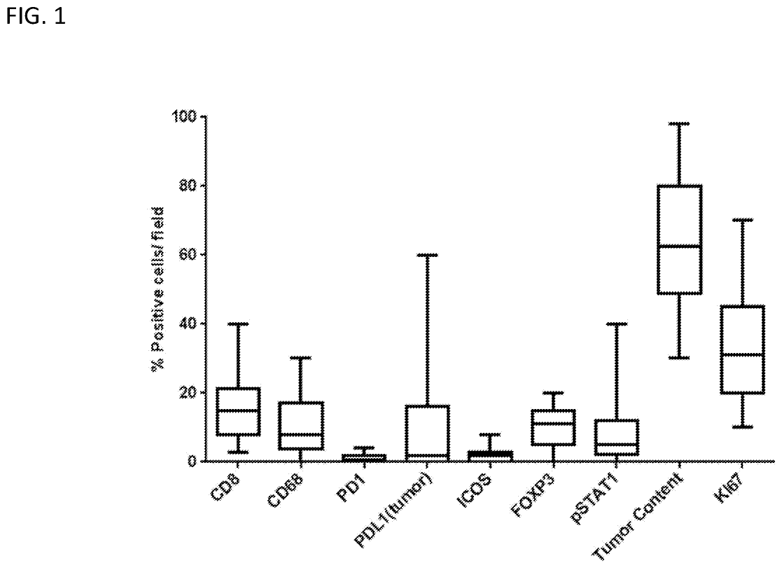

Patient tumors at baseline (TO, prior to culturing in the tumor microenvironment platform) were first evaluated for proliferation by Ki67 expression, tumor content by hematoxylin and eosin (H&E) staining, and expression of markers including CD8, CD68, PD-1, PD-L1, ICOS, FOXP3, and pSTAT1. Expression was measured by immunohistochemistry (IHC) using antibodies specific for the individual markers, and results are shown in FIG. 1 as box plots for percent of cells positive for each marker to highlight the variability between patient samples. Linear regression analysis was performed for several pairs of markers to probe for interrelationships, and results are shown in Table 1. None of the tested marker pairs showed significant linear correlation.

TABLE 1

| Marker Pair | R² |
|---|---|
| H&E vs Ki67 | 0.18 |
| CD8 vs Ki67 | 0.001 |
| CD8 vs FOXP3 | 0.08 |
| FOXP3 vs PD-Ll | 0.027 |
| PD-L1 vs CD68 | 0.06 |

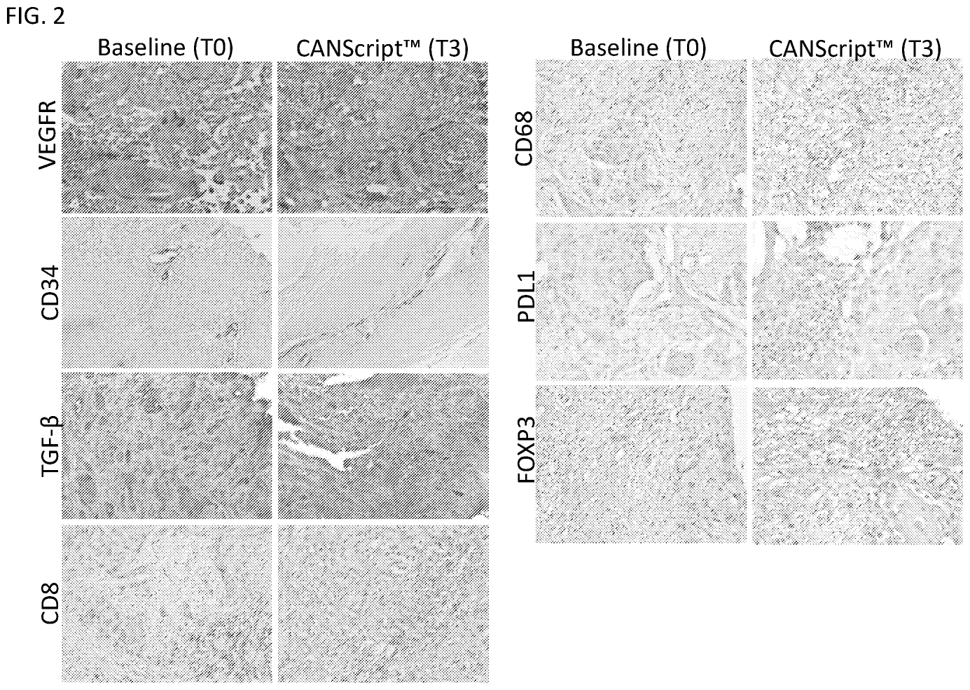

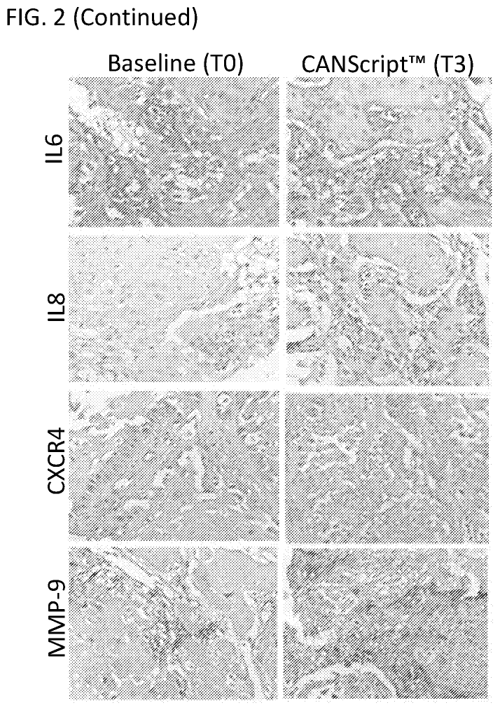

To demonstrate that the tumor microenvironment platform retains the markers of immune response tumor sections from patients at baseline (TO) and tumor sections cultured 3 days in the tumor microenvironment platform (T3) were stained by IHC for VEGFR, CD34, TGF-0, CD8, CD68, PDL1, FOXP3, IL6, IL8, CXCR4, and MMP-9. As shown in FIG. 2, staining was similar between baseline and after culturing for 3 days in the tumor microenvironment platform for each of the markers tested.

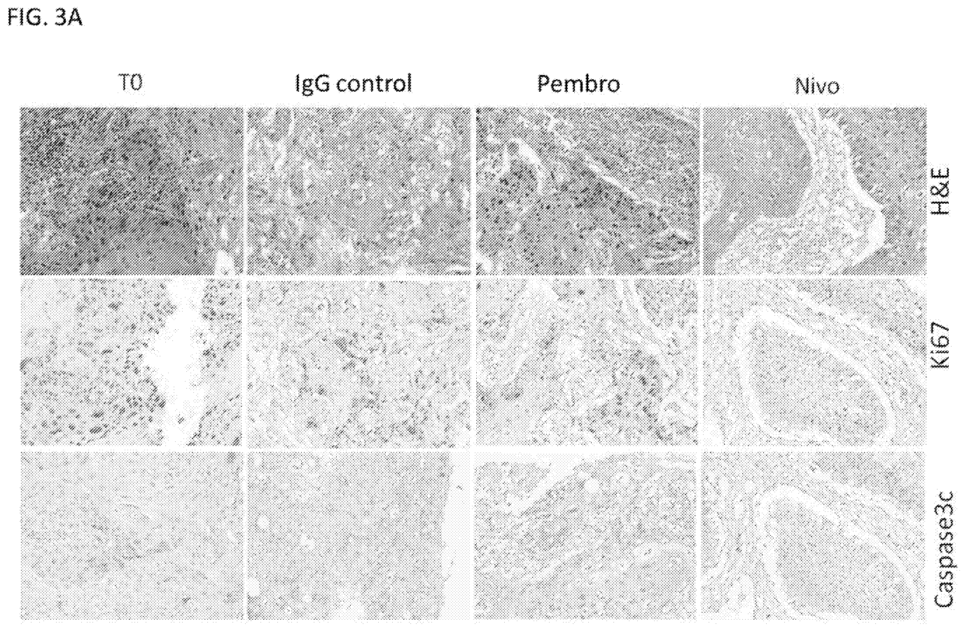

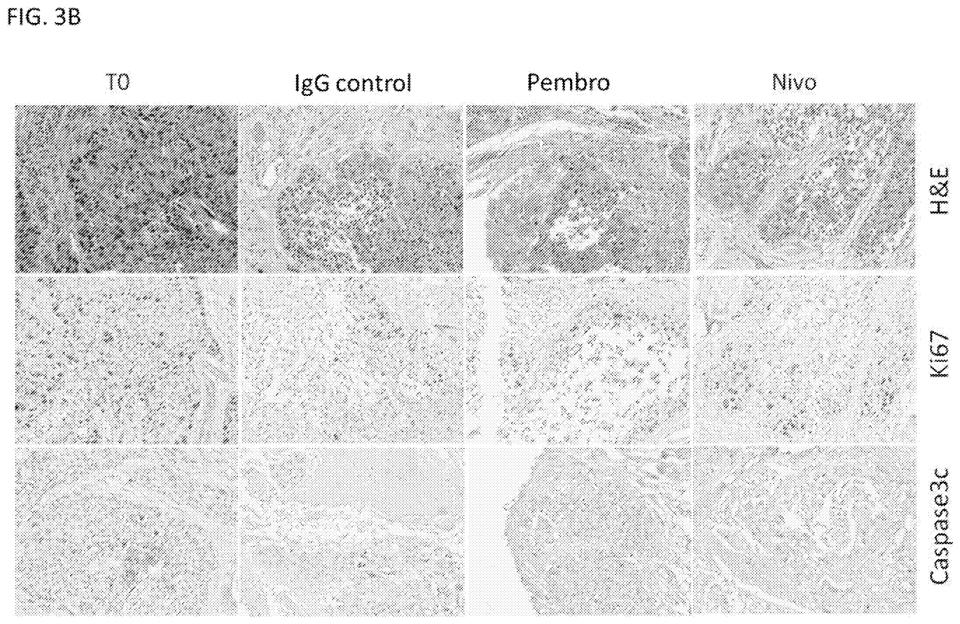

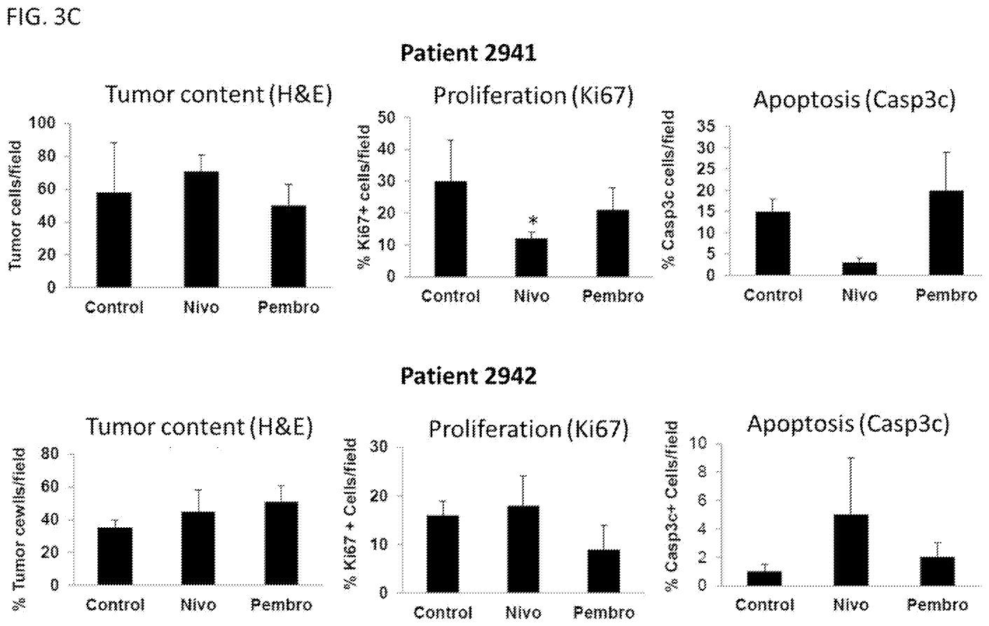

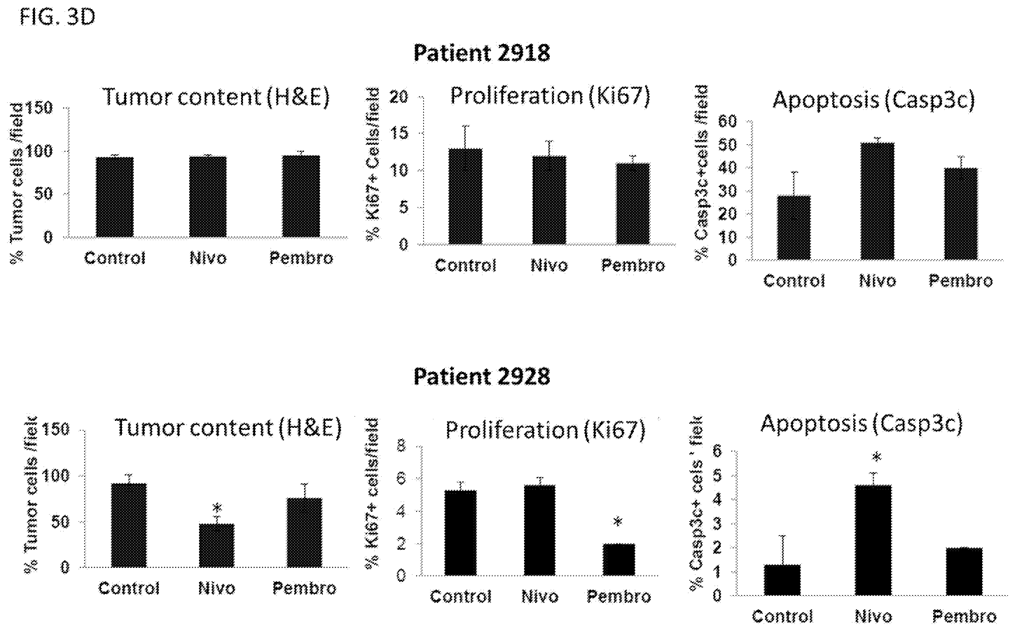

The effects of Pembrolizumab and Nivolumab on proliferation in the tumor microenvironment platform were then evaluated. Tumor sections from the same patient cultured for 72 hours in the tumor microenvironment platform (performed in at least biological triplicate) were treated with Pembrolizumab, Nivolumab, or IgG as control. Tissue was subsequently formalin fixed and paraffin embedded, and processed for H&E staining and immuno histochemical staining (IHC) with Ki67 and Caspase 3 at day 3 in culture (T3). Baseline staining was determined at TO. Results for two different patients, patient 2941 and patient 2942, are shown in FIGS. 3A and 3B, respectively, and quantified in FIG. 3C, and indicate that individual tumor sections obtained in at least biological triplicate can respond differentially to the anti-PD-1 antibodies with statistically significant deviation, as demonstrated by the diminished proliferation in response to Nivolumab but not Pembrolizumab in patient 2941 (FIGS. 3A and 3C), and similar differential antitumor effects in patient 2942 (FIGS. 3B and 3C). Results for two additional patients are quantified in FIG. 3D, further demonstrating differential anti-tumor responses to Pembrolizumab and Nivolumab in the same patient tumor.

The tumor microenvironment platform with tissue derived from each of the 16 patients and treated with either Pembrolizumab or Nivolumab was further evaluated using standard assays for tumor proliferation, tumor cell death, tumor morphology, and tumor cell viability as previously described, including tetrazolium salt WST-1 viability assay; LDH release; ATP uptake; glucose uptake; Caspase 3, Caspase 8, and Ki67 expression; and H&E staining. The results of the assays were used to generate assessment scores that were input into a machine-trained algorithm to generate a clinical outcome predictor in the form of an “M-score” for each patient for Pembrolizumab and Nivolumab, as shown in Table 2.

TABLE 2

| Patient | Nivolumab | Pembrolizumab |
|---|---|---|
| ID | (M-score) | (M-score) |
| 2916 | 13 | 8 |
| 2918 | 27 | 13 |
| 2926 | 31 | 28 |
| 2928 | 19 | 16 |
| 2939 | 5 | 4 |
| 2941 | 41 | 38 |
| 2942 | 28 | 18 |
| 2948 | 12 | 4 |
| 2949 | 11 | 6 |
| 2950 | 16 | 13 |
| 2956 | 10 | 6 |
| 2963 | 5 | 9 |
| 2978 | 33 | 22 |
| 2979 | 9 | 27 |
| 2980 | 12 | 19 |
| 2981 | 6 | 7 |

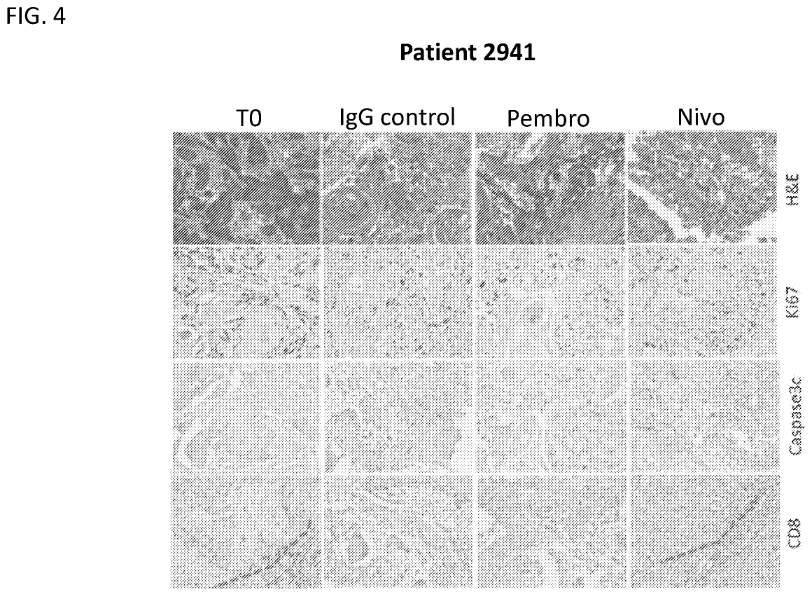

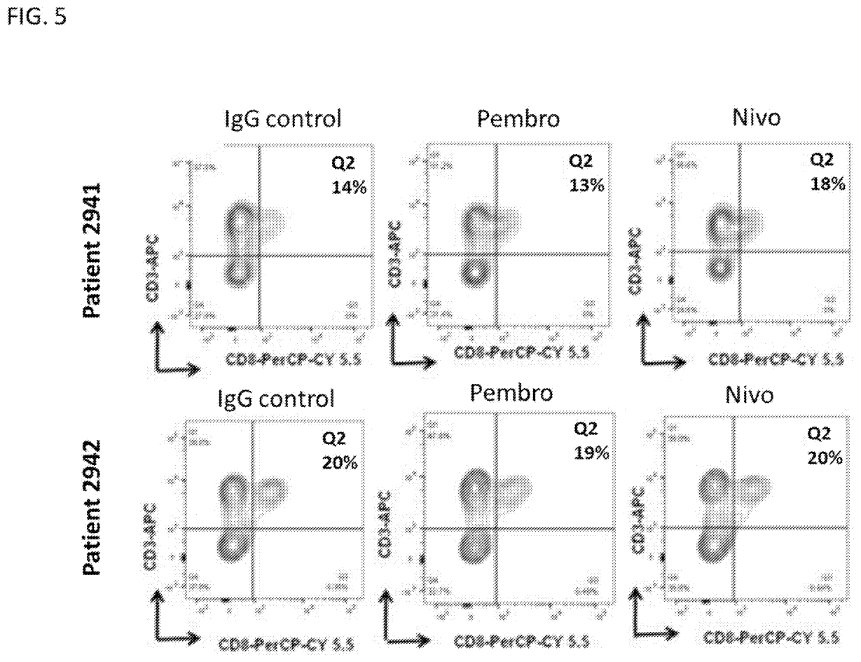

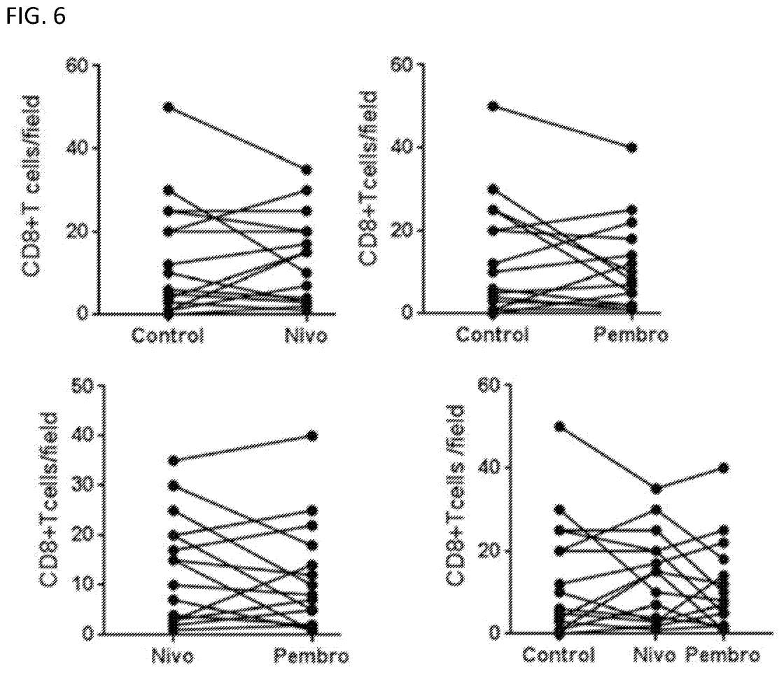

The effect of Pembrolizumab and Nivolumab on CD8⁺ T cell tumor infiltration was then evaluated. Tumor sections from the same patient cultured for 72 hours in the tumor microenvironment platform were treated with Pembrolizumab, Nivolumab, or IgG as control and stained for H&E, Ki67, Caspase 3, and CD8 at day 3 in culture (T3). Baseline staining was determined at TO. Results for one patient are shown in FIG. 4. Patient 2941 showed differentially modulated/induced CD8⁺ T cell infiltration with Nivolumab compared with Pembrolizumab. The increased CD8⁺ T cell infiltration with Nivolumab was associated with increased Caspase 3 activity. To further elucidate the effects of the anti-PD-1 antibodies, FACS analysis was performed on tumor tissue from patient 2941 and patient 2942 treated with either Pembrolizumab, Nivolumab, or IgG control. As shown in FIG. 5, there was an increase in CD8⁺ T cell infiltration for patient 2941 when treated with Nivolumab, but not Pembrolizumab, in agreement with the IHC results. By contrast, there was no effect of either Pembrolizumab or Nivolumab on CD8⁺ T cell infiltration for patient 2942, further highlighting the differential response between individuals to PD-1 blockade. CD8⁺ T cell infiltration was evaluated in the tumor microenvironment platform with tumor tissue from the same patient, and comparisons for control vs Nivo, control vs Pembro, Nivo vs Pembro, and control vs Nivo vs Pembro for multiple patients are shown in FIG. 6 (each line represents results from the tumor microenvironment platform cultured with tumor tissue from a single individual). These results provide further evidence for the heterogeneity in response between and within individuals to Pembrolizumab and Nivolumab that can be detected using the tumor microenvironment platform.

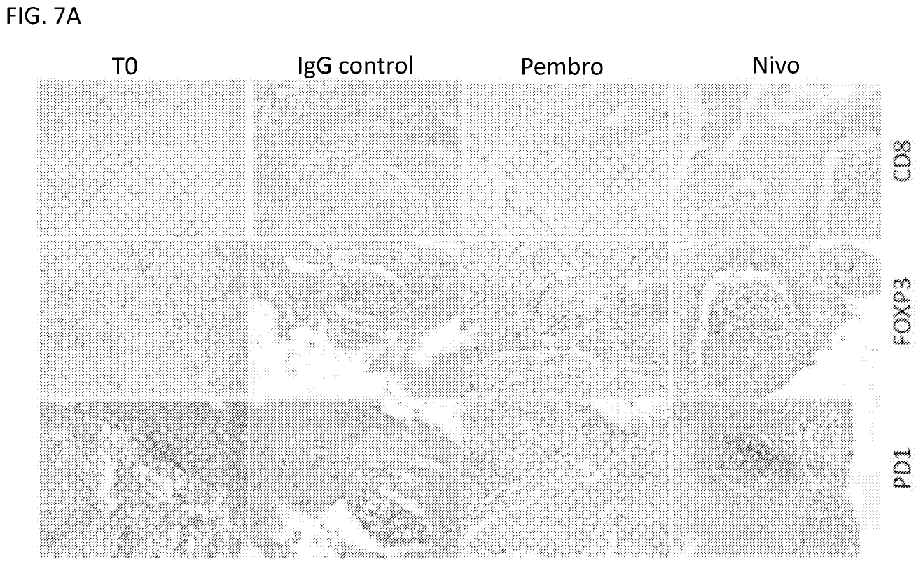

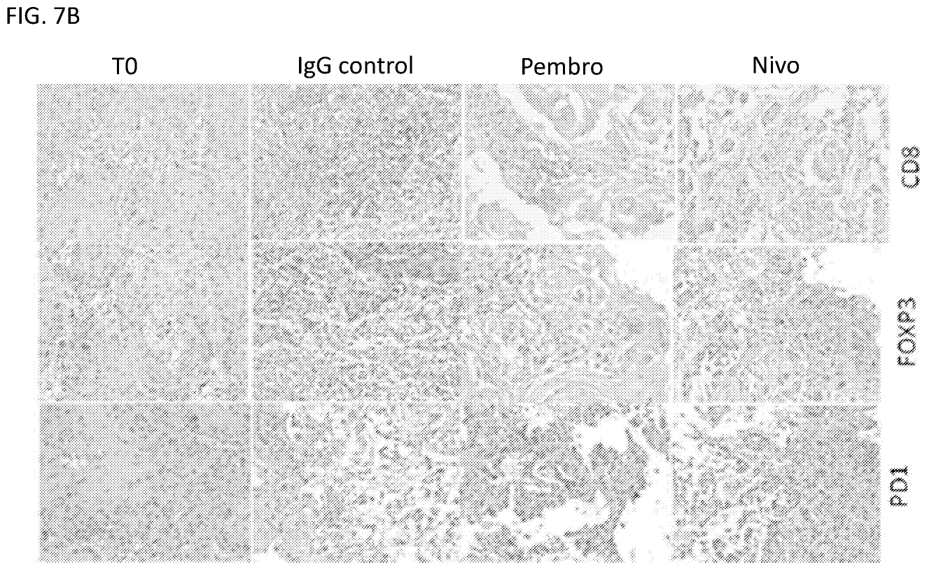

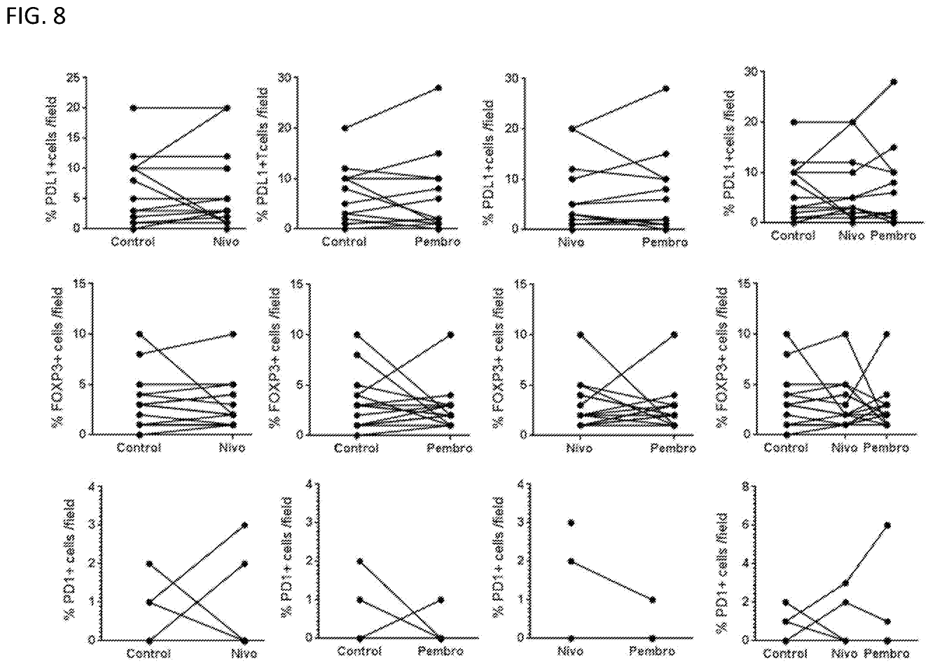

The effect of Pembrolizumab and Nivolumab on the expression of immunoregulatory molecules, including PD-1 and FOXP3, in the tumor microenvironment platform tumor microbed was evaluated. Tumor sections from the same patient cultured for 3 days (72 hours) in the tumor microenvironment platform were treated with Pembrolizumab, Nivolumab, or IgG control and stained for CD8, FOXP3, and PD-1 at day 3 in culture (T3). Baseline staining was determined at TO. Results are shown in FIG. 7A for tumor sections from a predicted responder to Pembrolizumab or Nivolumab and FIG. 7B for a predicted non-responder to Pembrolizumab or Nivolumab, as characterized by M-score. There was in increase in the number of PD-1⁺ cells in the tumor microbed for both Pembrolizumab and Nivolumab, which was to a greater degree in the case of Nivolumab, when compared to IgG control in the responder (FIG. 7A). There was no change for either Pembrolizumab and Nivolumab in the non-responder (FIG. 7B). The numbers of PD-L1⁺ tumor cells, PD-1⁺ T cells, FOXP3⁺ T-regulatory cells, and CD8⁺ T cells were evaluated using the tumor microenvironment platform with tumor tissue from the same patient in Nivolumab, Pembrolizumab, and control conditions, and are summarized in Table 3. Comparisons for control vs Nivo, control vs Pembro, Nivo vs Pembro, and control vs Nivo vs Pembro for multiple patients are shown in FIG. 8 (each line represents results from the tumor microenvironment platform cultured with tumor tissue from a single individual). The results show that using the tumor microenvironment platform, differential changes in expression of these regulatory molecules inside the tumor bed in response to Pembrolizumab and Nivolumab can be observed.

TABLE 3

| | # cells/field (Control; Nivo, Pembro) | | | | | | | | | | | |
|---|---|---|---|---|---|---|---|---|---|---|---|---|
| Patient ID | CD8⁺ | | | PD-L1⁺ | | | PD-1⁺ | | | FOXP3⁺ | | |
| 2916 | 20 | 30 | 18 | 1 | 2 | 2 | 0 | 0 | 0 | 3 | 4 | 2 |
| 2918 | 10 | 3 | 14 | 10 | 20 | 10 | 2 | 0 | 0 | 3 | 2 | 3 |
| 2926 | 25 | 20 | 5 | 3 | 3 | 1 | 0 | 0 | 0 | 4 | 3 | 10 |
| 2928 | 4 | 15 | 1 | 0 | 3 | 1 | 0 | 0 | 0 | 4 | 5 | 1 |
| 2939 | 0 | 2 | 5 | 10 | 10 | 15 | 0 | 0 | 0 | 1 | 2 | 1 |
| 2941 | 5 | 3 | 7 | 3 | 5 | 6 | 0 | 2 | 1 | 3 | 2 | 4 |
| 2942 | 3 | 1 | 2 | 0 | 0 | 0 | 0 | 0 | 0 | 3 | 4 | 2 |
| 2948 | 20 | 20 | 25 | 10 | 1 | 1 | 1 | 0 | 0 | 1 | 1 | 3 |
| 2949 | 50 | 35 | 40 | 20 | 20 | 28 | 1 | 3 | 6 | 5 | 5 | 3 |
| 2956 | 1 | 7 | 1 | 2 | 3 | 0 | 0 | 0 | 0 | 8 | 10 | 1 |
| 2963 | 6 | 4 | 2 | 12 | 12 | 10 | 0 | 0 | 0 | 10 | 2 | 2 |
| 2978 | 12 | 17 | 22 | 1 | 1 | 1 | 0 | 0 | 0 | 1 | 1 | 2 |
| 2979 | 25 | 25 | 10 | 5 | 5 | 8 | 0 | 0 | 0 | 2 | 1 | 3 |
| 2980 | 30 | 10 | 8 | 8 | 1 | 2 | 0 | 0 | 0 | 1 | 1 | 2 |
| 2981 | 1 | 15 | 12 | 0 | 0 | 0 | 0 | 0 | 0 | 0 | 1 | 1 |

To further probe the phenotypic modulation mediated by treatment with Nivolumab, tumor sections or PBNCs from the same patient cultured for 3 days (72 hours) in the tumor microenvironment platform were treated with Nivolumab or IgG control for one day, followed by flow cytometry gating for lymphocytes based on their forward and side scatter properties. Lymphocytes were further gated for expression of both CD3 and CD45, and this population of cells was analyzed by FACS for expression of PD-1, CEACAM, LAG3, TIM3, OX40, ILDR2, 4-1-BB, and GITR. Results are summarized in Table 4. Treatment with Nivolumab in the tumor microenvironment platform containing tumor tissue resulted in a decrease in the number of ILDR2⁺/CD3⁺/CD45⁺ lymphocytes and an increase in the number of GITR⁺/CD3⁺/CD45⁺ lymphocytes. No significant change was observed for these cells populations in the tumor microenvironment platform containing only PBNCs.

TABLE 4

| | Tumor (% total cells) | | PBMCs (% total cells) | |
|---|---|---|---|---|
| Marker | Control | Nivolumab | Control | Nivolumab |
| PD-1 | 19.4 | 20.5 | 2.94 | 0.31 |
| CEACAM | 2.04 | 3.69 | 0.12 | 2.74 |
| LAG3 | 2.22 | 0.52 | 0.14 | 0.031 |
| TIM3 | 17.8 | 19.9 | 7.91 | 13.9 |
| ILDR2 | 24.4 | 17.1 | 1.96 | 2.67 |
| OX40 | 1.70 | 0 | 0.25 | 0 |
| 4-1-BB | 4.69 | 7.56 | 0.27 | 0.38 |
| GITR | 2.60 | 13.0 | 0.30 | 0.50 |

The effect of treatment with Nivolumab and Pembrolizumab on CD25, CD127, and FOXP3 expression was then evaluated. Tumor sections or PBNCs from the same patient cultured for 3 days in the tumor microenvironment platform were treated with Pembrolizumab, Nivolumab, or IgG control for one day followed by FACS analysis for expression of CD25, CD127, and FOXP3. Results are summarized in Table 5. Treatment with Nivolumab or Pembrolizumab in the tumor microenvironment platform containing tumor tissue resulted in patient sample-dependent functional Treg cell depression.

TABLE 5

| | Tumor (% total cells) | | | PBMCs (% total cells) | | |
|---|---|---|---|---|---|---|
| Marker | Control | Prem | Nivo | Control | Prem | Nivo |
| Responder | | | | | | |
| CD25⁻/CD127⁻ | 12.2 | 18.4 | 15.6 | 46.6 | 25.4 | 18.9 |
| CD25⁺/CD127⁻ | 23.0 | 17.7 | 25.4 | 11.7 | 7.77 | 6.53 |
| CD25⁻/CD127⁺ | 24.3 | 31.2 | 31.8 | 24.8 | 46.4 | 44.2 |
| CD25⁺/CD127⁺ | 40.5 | 32.6 | 27.2 | 16.8 | 20.4 | 30.4 |
| CD25⁻/FOXP3⁻ | 20.3 | 6.67 | 4.62 | 23.2 | 20.0 | 29.8 |
| CD25⁺/ | 25.7 | 5.83 | 5.20 | 3.04 | 3.94 | 8.91 |
| FOXP3⁻ | | | | | | |
| CD25⁻ | 18.9 | 42.5 | 45.7 | 52.1 | 54.0 | 37.3 |
| FOXP3⁺ | | | | | | |
| CD25⁺/ | 35.1 | 45.0 | 44.5 | 21.7 | 22.1 | 24.0 |
| FOXP3⁺ | | | | | | |
| Non-Responder | | | | | | |
| CD25⁻/CD127⁻ | 22.1 | 24.9 | 21.7 | 44.1 | 21.4 | 22.3 |
| CD25⁺/CD127⁻ | 25.4 | 24.3 | 22.1 | 15.1 | 7.71 | 8.22 |
| CD25⁻/CD127⁺ | 33.0 | 32.9 | 40.1 | 26.2 | 45.3 | 44.4 |
| CD25⁺/CD127⁺ | 19.6 | 17.8 | 16.1 | 14.6 | 25.6 | 25.1 |
| CD25⁻/FOXP3⁻ | 23.6 | 10.7 | 17.5 | 18.2 | 10.9 | 7.90 |
| CD25⁺/ | 6.52 | 5.03 | 6.45 | 3.67 | 2.8 | 1.40 |
| FOXP3⁻ | | | | | | |
| CD25⁻ | 35.5 | 48.4 | 46.5 | 56.0 | 57.2 | 61.7 |
| FOXP3⁺ | | | | | | |
| CD25⁺/ | 34.4 | 35.8 | 29.5 | 22.1 | 29.1 | 29.0 |
| FOXP3⁺ | | | | | | |

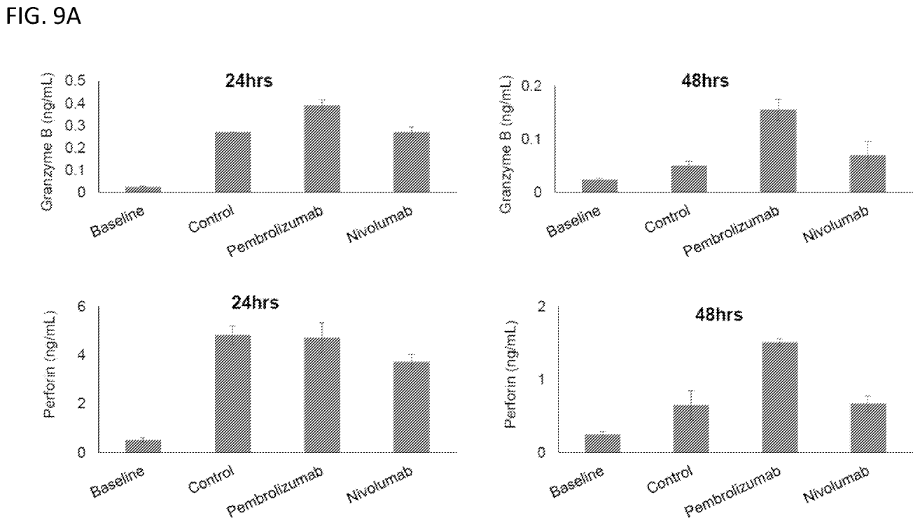

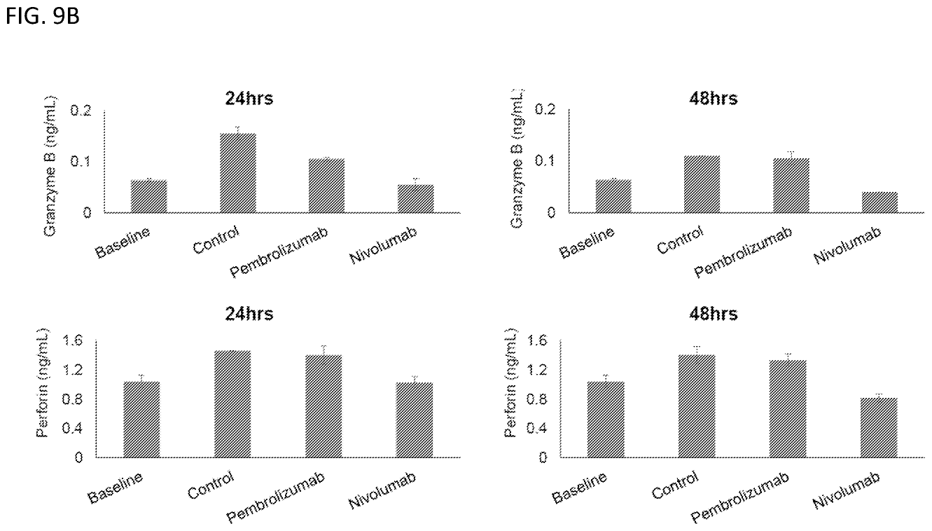

HNSCC tumor sections from the same patient cultured for 24 or 48 hours in the tumor microenvironment platform with Pembrolizumab, Nivolumab, or IgG as control were assayed for Granzyme B and Perforin secretion. Results are shown in FIG. 9A for the tumor microenvironment platform with tumor tissue from a predicted responder and FIG. 9B for the tumor microenvironment platform with tumor tissue from a predicted non-responder. After treatment with Pembrolizumab for 48 hours, there was an increase in both Granzyme B and Perforin secretion in the tumor microenvironment platform with tissue from the predicted responder compared to treatment with the control IgG. By contrast, at 48 hours in the tumor microenvironment platform with tissue from the predicted non-responder there was no increase in Granzyme B or Perforin secretion for treatment with either Pembrolizumab or Nivolumab.

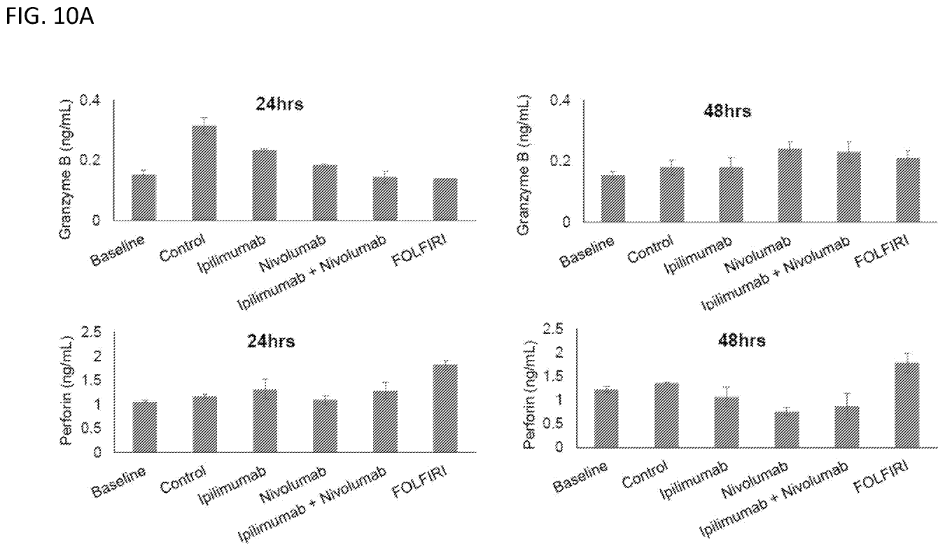

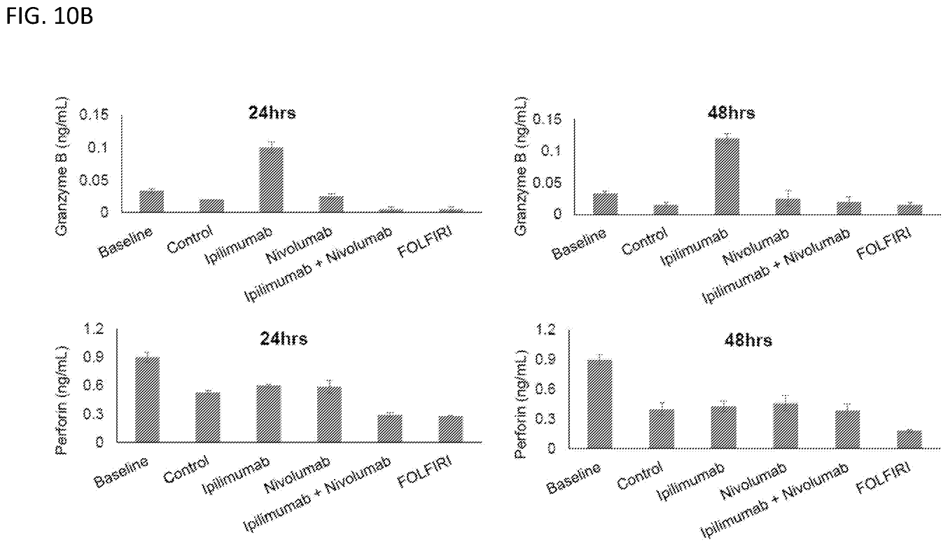

In another experiment, CRC tumor sections from the same patient cultured for 24 or 48 hours in the tumor microenvironment platform with Ipilimumab, Nivolumab, Ipilimumab+Nivolumab, FOLFIRI, or IgG as control were assayed for Granzyme B and Perforin secretion. Results are shown in FIG. 10A for the tumor microenvironment platform with tumor tissue from a predicted responder and FIG. 10B for the tumor microenvironment platform with tumor tissue from a predicted non-responder. After treatment with Nivolumab for 48 hours, there was an increase in Granzyme B secretion in the tumor microenvironment platform with tissue from the predicted responder compared to treatment with Ipilimumab or the control IgG. By contrast, at 48 hours in the tumor microenvironment platform with tissue from the predicted non-responder there was no increase in Granzyme B or Perforin secretion for treatment with Nivolumab.

The data demonstrate that the tumor microenvironment platform preserves the tumor-immune contexture and native heterogeneity across different clinical stages and patients. Importantly, we observed that PD-1 blockade resulted in patient-specific therapeutic response, which was characterized by differential distribution and maintenance of infiltrating CD8+ and CD4+ lymphocytes, distinct patterning of cytokines linked to functional dysregulation, and suppression of tumor proliferation and apoptosis. Interestingly, we determined that Pembrolizumab and Nivolumab induce functionally distinct mechanisms in the immune compartment, and disparate antitumor effects within an individual patient tumor.

Together, these findings demonstrate the utility of the tumor microenvironment platform as an ex vivo tool to predict therapeutic response to immune checkpoint inhibitors at the individual patient level. They also highlight that distinct mechanisms may contribute to clinical response of immune checkpoint inhibitors having the same molecular target. Such information can re-shape our understanding of personalized cancer care, and may impact rational treatment options in the emerging era of immunotherapy.

### EXEMPLARY EMBODIMENTS

### Embodiment 1

A method of predicting responsiveness to an immunotherapeutic agent for treating cancer in an individual in need thereof, the method comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform;
b) inputting the readout into a predictive model;
c) using the predictive model to generate an output; and
d) using the output to predict responsiveness of the individual to administration of the immunotherapeutic agent.

### Embodiment 2

A method of classifying likely responsiveness to an immunotherapeutic agent for treating cancer in an individual in need thereof, comprising: a) obtaining a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform;
b) inputting the readout into a predictive model;
c) using the predictive model to generate an output; and
d) using the output to classify the likely responsiveness of the individual to administration of the immunotherapeutic agent.

### Embodiment 3

A computer-implemented method for predicting responsiveness to an immunotherapeutic agent for treating cancer in an individual in need thereof, the method comprising:
a) accessing a readout comprising an assessment score for each of a plurality of assays conducted on a tumor tissue culture treated with the immunotherapeutic agent, wherein the tumor tissue culture comprises a tumor tissue from the individual cultured on a tumor microenvironment platform;
b) inputting the readout into a predictive model;
c) using the predictive model to generate an output; and
d) using the output to predict responsiveness of the individual to administration of the immunotherapeutic agent.

### Embodiment 4

The method of any one of embodiments 1-3, wherein the predictive model comprises an algorithm that uses each of the assessment scores as input and generates the output.

### Embodiment 5

The method of embodiment 4, wherein the algorithm comprises multiplying each of the input assessment scores with a corresponding weightage coefficient to obtain a plurality of weighted assessment scores; and combining the plurality of weighted assessment scores to generate the output.

### Embodiment 6

The method of any one of embodiments 1-5, wherein the output predicts complete clinical response, partial clinical response, or no clinical response of the individual to administration of the immunotherapeutic agent.

### Embodiment 7

The method of any one of embodiments 1-5, wherein the output predicts response or no response of the individual to administration of the immunotherapeutic agent.

### Embodiment 8

The method of any one of embodiments 1-7, wherein the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

### Embodiment 9

The method of any one of embodiments 1-8, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

### Embodiment 10

The method of embodiment 9, wherein the tumor microenvironment platform further comprises serum, plasma, and/or peripheral blood nuclear cells (PBNCs).

### Embodiment 11

The method of embodiment 10, wherein one or more of the serum, plasma, and/or PBNCs are derived from the individual.

### Embodiment 12

The method of any one of embodiments 1-11, wherein step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform.

### Embodiment 13

The method of any one of embodiments 1-12, wherein the assessment scores are generated based on a comparison between i) the results of the plurality of assays conducted on the tumor tissue culture treated with the immunotherapeutic agent; and ii) the results of the plurality of assays conducted on a reference tumor tissue culture, wherein the reference tumor tissue culture comprises a tumor tissue from the individual cultured on the tumor microenvironment platform.

### Embodiment 14

The method of embodiment 13, wherein the reference tumor tissue culture is not treated with the immunotherapeutic agent.

### Embodiment 15

The method of embodiment 13 or 14, wherein step a) further comprises conducting the plurality of assays on the reference tumor tissue culture; and/or step a) further comprises preparing the reference tumor tissue culture by culturing tumor tissue from the individual on the tumor microenvironment platform.

### Embodiment 16

A method of selecting a preferred therapeutic agent for treating cancer in an individual in need thereof from among a plurality of therapeutic agents against the same target molecule, the method comprising:
a) obtaining a readout comprising an assessment score for each of a plurality of assays conducted on tumor tissue cultures treated individually with each of the plurality of therapeutic agents, wherein the tumor tissue cultures each comprises a tumor tissue from the individual cultured on a tumor microenvironment platform;
b) inputting the readout into a predictive model;
c) using the predictive model to generate an output for each of the plurality of therapeutic agents;
d) using the outputs to predict responsiveness of the individual to administration of each of the plurality of therapeutic agents, and
e) selecting from among the plurality of therapeutic agents the therapeutic agent with the highest predicted responsiveness as the preferred therapeutic agent.

### Embodiment 17

The method of embodiment 16, wherein the predictive model comprises an algorithm that, for each of the plurality of therapeutic agents, uses each of the assessment scores for the given therapeutic agent as input and generates the output for the given therapeutic agent.

### Embodiment 18

The method of embodiment 17, wherein the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent.

### Embodiment 19

The method of any one of embodiments 16-18, wherein the output for a given therapeutic agent predicts complete clinical response, partial clinical response, or no clinical response of the individual to administration of the given therapeutic agent.

### Embodiment 20

The method of any one of embodiments 16-18, wherein the output for a given therapeutic agent predicts response or no response of the individual to administration of the given therapeutic agent.

### Embodiment 21

The method of any one of embodiments 16-20, wherein the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

### Embodiment 22

The method of any one of embodiments 16-21, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

### Embodiment 23

The method of embodiment 22, wherein the tumor microenvironment platform further comprises serum, plasma, and/or peripheral blood nuclear cells (PBNCs).

### Embodiment 24

The method of embodiment 23, wherein one or more of the serum, plasma, and/or PBNCs are derived from the individual.

### Embodiment 25

The method of any one of embodiments 16-24, wherein step a) further comprises conducting the plurality of assays on the tumor tissue cultures, thereby obtaining the readout comprising assessment scores from the plurality of assays, and/or step a) further comprises preparing the tumor tissue cultures by culturing tumor tissue from the individual in the tumor microenvironment platform.

### Embodiment 26

The method of any one of embodiments 16-25, wherein the assessment scores for a given therapeutic agent are generated based on a comparison between i) the results of the plurality of assays conducted on the tumor tissue culture treated with the given therapeutic agent; and ii) the results of the plurality of assays conducted on a reference tumor tissue culture, wherein the reference tumor tissue culture comprises a tumor tissue from the individual cultured on the tumor microenvironment platform.

### Embodiment 27

The method of embodiment 26, wherein the reference tumor tissue culture is not treated with any of the plurality of therapeutic agents.

### Embodiment 28

The method of embodiment 26 or 27, wherein step a) further comprises conducting the plurality of assays on the reference tumor tissue culture; and/or step a) further comprises preparing the reference tumor tissue culture by culturing tumor tissue from the individual on the tumor microenvironment platform.

### Embodiment 29

A method of treating cancer in an individual in need thereof, the method comprising administering to the individual an immunotherapeutic agent to which the individual is predicted to respond according to the method of any one of embodiments 1-15 that predicts responsiveness.

### Embodiment 30

The method of embodiment 29, wherein the individual is predicted to have a complete clinical response or partial clinical response to administration of the immunotherapeutic agent.

### Embodiment 31

A method of treating cancer in an individual in need thereof, the method comprising administering to the individual a preferred therapeutic agent from among a plurality of therapeutic agents against the same target molecule, wherein the preferred therapeutic agent is selected according to the method of any one of embodiments 16-28.

### Embodiment 32

The method of embodiment 31, wherein the individual is predicted to have a complete clinical response or partial clinical response to administration of the preferred therapeutic agent.

### Embodiment 33

The method of any one of embodiments 1-15, 29, and 30, wherein the immunotherapeutic agent is an immune checkpoint inhibitor.

### Embodiment 34

The method of embodiment 33, wherein the immune checkpoint inhibitor is an antagonistic antibody targeting an immune checkpoint molecule.

### Embodiment 35

The method of embodiment 33 or 34, wherein the immune checkpoint inhibitor is pembrolizumab or nivolumab.

### Embodiment 36

The method of any one of embodiments 16-28, 31, and 32, wherein the plurality of therapeutic agents comprises a plurality of immune checkpoint inhibitors.

### Embodiment 37

The method of embodiment 36, wherein the plurality of immune checkpoint inhibitors comprises a plurality of antagonistic antibodies targeting an immune checkpoint molecule.

### Embodiment 38

The method of embodiment 36 or 37, wherein the plurality of immune checkpoint inhibitors comprises pembrolizumab and nivolumab.

1-38. (canceled)

39. A method of selecting a therapeutic agent for treating cancer in an individual in need thereof from among a plurality of therapeutic agents against the same target molecule, the method comprising:
a) obtaining a readout comprising an assessment score for each of a plurality of assays conducted on tumor tissue cultures treated individually with each of the plurality of therapeutic agents,
wherein the tumor tissue cultures each comprises a tumor tissue from the individual cultured on a tumor microenvironment platform;
b) inputting the readout into a computer comprising a non-transitory, computer-readable program code comprising a predictive model;
c) using the predictive model to generate an output for each of the plurality of therapeutic agents; and
d) using the outputs to predict responsiveness of the individual to administration of each of the plurality of therapeutic agents

40. The method of claim 39, further comprising the step of: e) selecting from among the plurality of therapeutic agents the therapeutic agent with the highest predicted responsiveness as the therapeutic agent.

41. The method of claim 39, wherein the predictive model comprises an algorithm, that for each of the plurality of therapeutic agents uses each of the assessment scores for the given therapeutic agent as input and generates the output for the given therapeutic agent.

42. The method of claim 41, wherein the algorithm comprises, for each of the plurality of therapeutic agents, multiplying each of the input assessment scores with a corresponding weightage coefficient to obtain a plurality of weighted assessment scores; and combining the plurality of weighted assessment scores to generate the output for the given therapeutic agent.

43. The method of any one of claim 42, wherein the output for a given therapeutic agent predicts complete clinical response, partial clinical response, or no clinical response of the individual to administration of the given therapeutic agent.

44. The method of any one of claim 42, wherein the output for a given therapeutic agent predicts response or no response of the individual to administration of the given therapeutic agent.

45. The method of claim 39, wherein the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

46. The method of claim 39, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

47. The method of claim 46, wherein the tumor microenvironment platform comprises one or more of serum, plasma, and peripheral blood nuclear cells (PBNCs).

48. The method of claim 47, wherein one or more of serum, plasma, and PBNCs are obtained from the individual.

49. The method of claim 39, wherein the plurality of therapeutic agents targets an immune checkpoint molecule.

50. The method of claim 39, wherein the plurality of therapeutic agents targets a PD-1 protein.

51. The method of claim 50, wherein the plurality of therapeutic agents are anti-PD-1 antibodies.

52. The method of claim 51, wherein the anti-PD-1 antibodies are nivolumab and pembrolizumab.

53. The method of claim 39, further comprising treating the individual with the therapeutic agent which has the highest predicted responsiveness.

54. A method of treating an individual, comprising: a) collecting tumor tissue from said individual; b) having responsiveness of a plurality of therapeutic agents determined; and c) treating said individual with a therapeutic agent which has the highest predicted responsiveness;
wherein said having responsiveness of a plurality of therapeutic agents determined comprises: receiving outputs of predicted responsiveness of the individual to administration of each of the plurality of therapeutic agents, said outputs determined by use of a predictive model to generate an output for each of the plurality of therapeutic agents, said predictive model having assessed readouts comprising an assessment score for each of a plurality of assays conducted on tumor tissue cultures of said tumor tissue treated individually with each of the plurality of therapeutic agents, said tumor tissue cultures cultured on a tumor microenvironment platform.

55. The method of claim 54, wherein the plurality of assays is selected from the group consisting of cell viability assays, cell death assays, cell proliferation assays, tumor morphology assays, tumor stroma content assays, cell metabolism assays, senescence assays, cytokine profile assays, enzyme activity assays, tumor and/or stromal cell expression assays, and any combination thereof.

56. The method of claim 54, wherein the tumor microenvironment platform comprises an extracellular matrix composition comprising one or more of collagen 1, collagen 3, collagen 4, collagen 6, Fibronectin, Vitronectin, Cadherin, Filamin A, Vimentin, Osteopontin, Laminin, Decorin, and Tenascin C.

57. The method of claim 56, wherein the tumor microenvironment platform comprises one or more of serum, plasma, and peripheral blood nuclear cells (PBNCs).

58. The method of claim 57, wherein one or more of serum, plasma, and PBNCs are obtained from the individual.

59. The method of claim 54, wherein the plurality of therapeutic agents targets an immune checkpoint molecule.

60. The method of claim 44, wherein the plurality of therapeutic agents targets a PD-1 protein.

61. The method of claim 60, wherein the plurality of therapeutic agents are anti-PD-1 antibodies.

62. The method of claim 61, wherein the anti-PD-1 antibodies are nivolumab and pembrolizumab.
